(19) United States
(12) Patent Application Publication
(10) Pub. No.: US 2007/0141071 A1
(43) Pub. Date: Jun. 21, 2007

(54) Hot melt coating by direct blending and coated substrates

(75) Inventor: James W. Ayres, Corvallis, OR (US)

Correspondence Address: KLARQUIST SPARKMAN, LLP, 121 SW SALMON STREET, SUITE 1600, PORTLAND, OR, 97204, US

(73) Assignee: Oregon State University

(21) Appl. No.: 11/698,223

(22) Filed: Jan. 24, 2007

Related U.S. Application Data
(62) Division of application No. 10/438,786, filed on May 14, 2003.

(51) Int. Cl. A61K 39/00

(52) U.S. Cl. 424/184.1

(57) ABSTRACT

A method of coating a pharmaceutical substrate which is not a hot-melt coating by fluid bed method comprises applying a molten coating material to the pharmaceutical substrate wherein the substrate is coated with the coating material; optionally applying to the coated substrate the same or different molten coating material, and optionally repeating the second applying step; wherein the coated substrate contains an antigen or a pharmaceutical agent or drug; and wherein the molten coating contains less than 10% solvent. Coated substrates include those made by this process.

### FIELD OF THE INVENTION

The invention is directed to a method of hot-melt coating pharmaceutical substrates that utilizes direct blending of molten coating material with the substrate. In addition, the invention includes coated substrates that can include pharmaceutical agents, as well as methods of using the coated substrates. Coated pharmaceutical substrates comprising heat sensitive antigens prepared by direct blending of molten coating material with heat sensitive antigens are included.

### BACKGROUND ART

Many early coating processes, such as pan coating, required the use of large amounts of solvents. In addition, conventional solvent-based, fluid-bed based coating processes have been widely applied for formulation and development of many pharmaceutical dosage forms. These coating methods require solvents for solution or dispersion preparation, which involve using polymers and organic solvents to produce the desired coatings on a substrate fluidized on a bed or in a column of air. However, the introduction of the Clean Air Act in 1970 by the U.S. Environment Protection Agency (EPA) has since been dictating control of organic solvents in the pharmaceutical industry and prompting new alternatives to the application of organic solvents. Although still receiving relatively little attention, hot-melt coating systems have become an area in the pharmaceutical industry where more and more research effort has been applied to develop alternatives to organic- or aqueous-based polymer systems.

One conventional approach is hot-melt coating by fluid bed spraying. In this hot melt coating process the coating material is applied onto the substrate surface in the molten state, providing several advantages over the current and conventional coating techniques that use dissolved or suspended polymers. One advantage to this conventional approach is that hot melt coating negates the use of solvent and avoids recovery/treatment of organic solvents. Water, a cause of drug degradation during processing, may be avoided as well. In addition, processing times may be reduced. One purpose of hot melt coating is in controlling drug release from pharmaceutical dosage forms.

Development of hot melt coating methods is not without challenges. These include the thermal degradation effect of the hot melt coating material in the molten state on the drug substances in the formulation, significant limitations in the rate or speed at which melt coatings that can be applied, and an essential need for characterization of complex coating equipment required in carrying out such processes.

Early work on development of hot melt coating methods was conducted by employing standardized fluidized bed (top spray) for coating small pellets, granules, and particles using molten materials. Spraying molten wax onto particles in a fluidized bed was used for drug encapsulation. A controlled drug release dosage form was produced by spraying lipid matrix agent over individual grains comprised of drug and adjuvant particles.

A conventional hot melt spray coating process carried out with a fluidized bed may consist of three steps: spraying of molten material onto substrate surface while maintaining constant substrate fluidization with a stream of air, spreading of the molten material around the substrate surface in a fluidized bed, and congealment of the molten material while keeping the substrate fluidized. In order to prevent molten coating material from congealing prior to being delivered to the substrate surface, the coating is normally kept at a temperature of 40-60° C. above its melting point. To maintain constant temperature, atomization air has to be heated to the same level as the molten coating material. Also, the nozzle needle must be insulated to prevent re-melting of the congealed molten coating material.

Another conventional process is solid dispersion hot-melt coating in a fluid bed. In this process, the solid dispersion hot-melt fluid bed coating method was applied in a fluidized bed with a Wurster bottom spray insert. This technique focused on hot-melt coating substrate by combining the coating agent and the substrate together in a fluid chamber bed. This system was simpler than the hot-melt coating method utilizing spraying technique with respect to the coating setup by eliminating the need for spraying the molten coating material onto the substrate surface.

This process has a number of disadvantages. For instance, one limitation in these procedures is that coating agents can only be used with melting points and molten viscosities less than 80° C. and 300 centipoise, respectively. It was shown that the maximum feasible hot-melt coating level can only be varied from 2.5 percent to 5.5 percent depending on different substrate sizes. Also, substrates of 10-30 U.S. standard mesh (0.5 to 2.0 mm) can be coated as individual particles, while particle sizes smaller than 40 mesh (0.42 mm) agglomerate. In addition, one drawback reported was that to maintain batch-to-batch reproducibility and overall robustness of the final product, seal coatings or strict substrate porosity specifications are required. For multiple coating, another problem is that melting points of multiple coating agents must differ by 15° C. or more.

As a result of these limitations, the solid dispersion hot-melt fluid bed coating method can only be applied selectively, and the relatively higher cost-effectiveness of this method cannot be justified with such a narrow application window. A need remains for a hot melt coating method that avoids the known problems of hot melt spray coating with a fluid bed, and also avoids the new problems of the newer solid dispersion hot-melt fluid bed coating method. In addition, a need remains for an improved process for making antigen-containing coated substrates.

Oral delivery for allergens has been achieved based on application of allergen onto nonpareils, and then enterically coating with methyl methacrylic copolymer as shown schematically in FIG. 1. Both the allergen application and the enteric coating layer has been achieved through the conventional fluidized bed (“Wurster”) spray coating process. However, this spray coating technique for the allergen application introduced problems such as a low efficiency of coating and long processing times. As a result, there is a need for a novel application technique that can overcome these problems. The present invention eliminates the need for spray-application of the pharmaceutical agent or allergen layer (FIG. 1) and reduces the processing time and thus production cost.

### DISCLOSURE OF THE INVENTION

To improve upon the currently existing hot-melt coating methods (those utilizing fluidized bed as spraying platform and the solid dispersion hot-melt fluid bed coating), a new hot-melt coating technique has been discovered and termed “hot-melt coating by direct blending.” Results from this method have demonstrated surprising success without producing significant degradation of antigens that may be included in the coated product.

In one embodiment, the invention is directed to a method of coating a pharmaceutical substrate which method is not a hot-melt coating by fluid bed method comprising applying a molten coating material to the pharmaceutical substrate wherein the pharmaceutical substrate is coated with the coating material; optionally applying to the coated substrate the same or different molten coating material, and optionally repeating the second applying step; wherein the coated substrate contains an antigen or pharmaceutical agent; and wherein the molten coating material contains less than 10% solvent. In another embodiment, this method may further comprise the step of spray film coating the coated substrate, for example, with an enteric coating. In a preferred embodiment, the antigen or pharmaceutical agent is a heat sensitive antigen that is not degraded during the applying step or steps. In another embodiment, the molten coating material has a temperature of less than 40° C. above the melting point of the coating material. In a preferred embodiment, the coated substrate comprises greater than 6% of the coating material by weight based on the weight of the pharmaceutical substrate and/or the molten viscosity of the coating material is more than 300 centipoise and/or the pharmaceutical substrate comprises beads smaller than 40 mesh. In yet another embodiment, the antigen or pharmaceutical agent is uniformly dispersed in the coating material.

The method of the invention may further comprise congealing the coating material on the pharmaceutical substrate; and separating agglomerates from the congealed coated substrates before optionally applying to the coated substrates additional molten coating material. In addition, the method of the invention may further comprise mixing the coating material during and/or after the first applying step or additional applying steps while the molten coating material is cooling and congealing on the substrate. In one embodiment, the mixing step comprises mechanical mixing of the pharmaceutical substrate with the molten coating material in a coating vessel and optionally scraping wall surfaces of the coating vessel. In other embodiment, the pharmaceutical substrate is preheated before the applying step.

In another embodiment, the invention is directed to a coated substrate prepared by the method described above. In another embodiment, the invention is directed to a coated substrate comprising a coating material coated on a pharmaceutical substrate; wherein the coated substrate contains at least one layer; wherein each of the at least one layer contains coating material having greater than 8% by weight of the weight of the pharmaceutical substrate; and optionally comprising an enteric coating on the coated substrate.

In another embodiment, the invention is directed to an immunogenic composition which induces an immunological response in a host subject inoculated with the composition comprising the coated substrate defined above and a method to induce an immune response in a subject against an allergic reaction, comprising administering to a subject in need thereof an effective amount of the immunogenic composition, preferably wherein the immunogenic composition is ragweed pollen extract.

The novel hot-melt coating method described above completely negates the requirement for a fluidized bed as spraying platform, thus reducing processing time, cutting production cost, and eliminating the disadvantages seen with the two hot-melt coating processes described above. In addition, a protein antigen has unexpectedly been shown to be stable when mixed in the molten coating process. Further, excellent uniformity is obtained, and maximal coating application greatly exceeds what is known in the art for solid dispersion hot-melt fluid bed coating without the reported particulate agglomeration. The rate of melt coating material application exceeds material spray application rate for fluid bed melt coating.

### BRIEF DESCRIPTION OF THE DRAWINGS

FIG. 1 shows a schematic diagram of structure of allergen encapsulation.

FIG. 2 is directed to a non-limiting embodiment of a 5-stage hot-melt coating process. Stage 1 includes hot melt agent melting and cooling; stage 2 includes antigen dispersion/dissolution in molten material; stage 3 includes nonpareils preheating; stage 4 includes mixing/blending of nonpareils with hot melt material; and stage 5 includes cooling and congealing of hot-melt on the nonpareil surface.

FIG. 3 shows comparison of drug uniformity test results at different amounts of Gelucire® 50/13 using Ovalbumin as model compound.

FIG. 4 shows drug content uniformity test results from Gelucire® 50/13 hot melt coated nonpareils with direct blending method using Ragweed Pollen Extract (RPE) as model compound.

FIG. 5 shows RPE release profile from enteric-coated Gelucire® 50/13 hot-melt coated nonpareils. The dissolution medium was 0.1 N HCl simulated gastric fluid (SGF) of pH˜1.29.

FIG. 6 shows RPE release profile from enteric-coated Gelucire® 50/13 hot-melt coated nonpareils. The dissolution medium was the simulated intestinal fluid (SIF) of pH˜6.8.

FIG. 7 shows dissolution profile in acid of RPE from Gelucire 50/13 hot-melt filled capsule.

FIG. 8 shows dissolution profile in base of RPE from Gelucire 50/13 hot-melt filled capsule.

FIG. 9 shows scanning electron micrographs of Gelucire® 50/13 hot-melt coated nonpareil. FIG. 9a shows the surface view (×100) and FIG. 9b shows the cross section view (×150).

FIG. 10 shows scanning electron micrographs of enteric-coated Gelucire® 50/13 hot-melt coated nonpareils containing RPE. FIG. 10a shows the surface view (×100) and FIG. 10b shows the cross section view (×150).

FIG. 11 shows a scanning electron micrograph of enteric-coated Gelucire® 50/13 hot-melt coated nonpareils having a cross section view (×150).

FIG. 12 shows differential scanning calirometry (DSC) thermograms comparing the melting points of Gelucire® 50/13 (FIG. 12a) and mixture of Gelucire® 50/13 of RPE (5:1) (FIG. 12b).

FIG. 13a is Gelucire50/13/stearic acid coated capsules (about 1000% in weight gain) before floating study; FIG. 13b is Gelucire50/13/stearic acid coated capsules after 16 hours of floating in simulated intestinal fluid. The floating study was conducted at room temperature under static condition.

FIG. 14 shows ELISA Inhibition assay results. All three profiles possess comparable slopes indicating similar binding between the anti-RPE antibodies and the RPE-inhibitor. Also drawn from examining the profiles is the conclusion that RPE remains in its native conformational form following encapsulation into microbeads by both the spray coat formulation and the direct blend formulation.

FIG. 15 shows averaged total serum anti-RPE antibody titers responsiveness for the treatment groups (Groups I and III) and the placebo treatment (Group II) after oral administration of RPE encapsulated enteric-coated microbeads.

FIG. 16 shows averaged total serum anti-RPE antibody titers for mice in the treatment groups (Groups I and III) and in the placebo treatment (Group II) after oral administration of RPE encapsulated enteric-coated microbeads. The data employed for the graph were only from mice in groups I and III that showed antibody responsiveness to RPE.

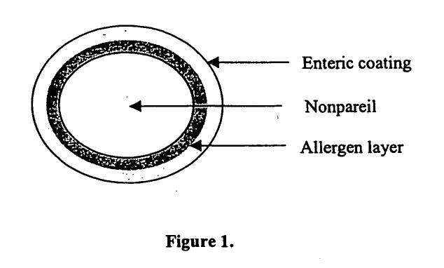

### MODES OF CARRYING OUT THE INVENTION

In the method of the invention or products made thereby, any pharmaceutically acceptable substrate can serve as a substrate or support. The substrate may encompass a variety of shapes and forms. Some non-limiting examples are micro-spheres, beads, granules, tablets, capsules, chewable wafers/tablets, matrix beads or nonpareils. These substrates may contain a pharmacologically active agent or may be pharmacologically inert. In one preferred embodiment the substrate is inert nonpareils that have a mesh size of 10-100 mesh, preferably 10-45, more preferably 30-45, and most preferably 35-45. In contrast to conventional methods, the method of the present invention allows much smaller beads to be used without agglomeration during the rapid application of melt coating in the process, relative to spray melt coating methods.

In one embodiment of a capsule, a preferred embodiment includes a capsule that is partially full. In another preferred embodiment, the substrate is an empty capsule, and preferably contains a pharmaceutical agent in the coating material. Empty capsules can be used as substrates for advantageously making capsules that float, for example, in the gastric fluids of the stomach. In another preferred embodiment, the capsule prepared by the hot-melt coating by direct blending method of the invention is capable of floating in simulated gastric fluid without enzymes for at least about 2 hours at 37° C. Further, it is preferred that the coated capsule swells at least about twice the original volume in a gastric fluid. In a more preferred embodiment, the substrate is a helium-filled capsule and the coating application step is conducted in a helium atmosphere.

One embodiment of the substrate comprises beads coated by the process of the invention and chewable tablets that may comprise such coated beads. The chewable tablets that provide sustained release of active pharmacological agents are very difficult to formulate. Typically, chewing destroys any formulation structure such as coating of a substrate which contains drug wherein the coating material influences drug release rate. In addition, compression of coated beads wherein the coating material controls drug release rate results in fracture and destruction of the coating material as a result of tablet compression forces. U.S. Pat. No. 5,766,623 entitled “Compactable Self-Sealing Drug Delivery Agents,” issued to Ayres, describes these problems in more detail. Clearly, there is a need to be able to apply sufficient quantities of coating material that is sufficiently flexible to deform and thereby resist tablet compression forces and chewing forces. Heretofore no such method has been available. However, based on Example 3 herein, it is expected that soft and flexible semisolid materials can be easily coated onto a pharmaceutical substrate such as nonpareil beads by using the method of the invention. In a preferred embodiment the chewable tablet of the invention is capable of providing sustained release of the pharmaceutical agent for at least about 4 hours.

The substrate is preferably preheated before submersion into the coating material, although heating is not required. For example, the substrate may be heated to the approximate temperature of the molten coating material, and at temperatures above or below the temperature of the molten coating material. Preferably, in the method of the invention, the substrate is heated to approximately the melting point of the coating material.

The substrate can be submerged, mixed, covered, or partially covered with a molten coating material in, for example, a vat or mixing chamber such that a coated substrate is produced when the molten coating material is cooled. The molten coating material is preferably applied without spraying but may be sprayed onto the substrate. In addition, the molten coating material can be added to the substrate, or the substrate can be added to the molten coating material. The application of the molten coating material of the present invention does not include the application of a coating onto a substrate using a spray melt coating method or a solid dispersion hot-melt fluid bed coating method. Spray melt coating in which melted coating material is sprayed onto a bed of substrate fluidized in a column of air is well understood by one skilled in the art to be known as spray melt coating and includes use of a Wurster column, as described in Jozwiekowski, R. M., et al., Pharmaceutical Research (1990) 7:1119-1126; Gabriel, R., et al. and U.S. Pat. No. 5,258,132. Solid dispersion hot-melt fluid coating methods include those in which the coating material is not sprayed onto the substrate but rather the coating material and substrate are combined in a fluidized bed and then as the coating material is heated, it melts onto the substrate. Kennedy, J. P., et al., Pharmaceutical Development and Technology (1996) 1:51-62.

In another preferred embodiment, coated discrete pharmaceutical substrates are produced by hot-melt coating by direct blending in that separate individual coated substrate units are produced, e.g., if capsules are the pharmaceutical substrate then coated individual capsules become discrete coated pharmaceutical substrates. If some agglomeration of discretely coated substrates occurs, the agglomerates are separated to produce discrete coated pharmaceutical substrates. Some other examples of pharmaceutical substrates include tablets, beads, and granules. In all cases the starting pharmaceutical substrates before hot-melt coating by direct blending may be complex or simple substrate units, e.g., empty or filled capsules, matrix or coated tablets, granules or beads. The composition produced comprises a melt coating on a discrete pharmaceutical substrate. The coating material may include multiple additives such as excipients or drugs and the additives may be soluble or insoluble in the coating material such that the coating may itself be a matrix but the pharmaceutical substrate being coated is coated as discrete units, e.g., capsules, beads or tablets can be hot-melt coated by direct blending with a mixture of a waxy material and a drug and excipients to produce individually discrete coated capsules, beads or tablets. Such discrete particles distinguish over other types of coated substrates that are not discretely coated particles such as those where a powder substrate is dispersed throughout a coating material.

The coating material for coating on the substrate includes any suitable coating. One preferred embodiment is polyethylene glycols (PEG) of various molecular weights, such as PEG 3350, and/or PEG 4600. Another preferred embodiment is Gelucire 50/13, which is a semi-solid bioavailability enhancer with surfactant activity. Other semisolid surfactants can also be used as a coating material, and liquid surfactants can be included as ingredients in preferred coating materials.

In addition, mixtures of coating materials can be used, such as a mixture of Gelucire 50/13 and stearic acid. In a preferred embodiment, the coating material or the combination of coating materials should be a semi solid or solid at room temperature.

In one embodiment the coating material preferred in the new process is a waxy or wax like material with a melting point of between about 30 degrees centigrade and about 90 degrees centigrade more preferably from about 45-65° C., and most preferably from about 50-55° C. Such materials include but are not limited to any of various individual or mixtures of natural or synthetic lipophilic or hydrophilic or surfactant polymeric materials that are solid, semisolid, or pliable at room temperature and after melting and then cooling produce a texture that is smooth, slippery, or waxy to the touch. Non-limiting examples include carbowaxes (polyethylene glycols) and derivatives thereof, surfactants, long-chain fatty acids and esters thereof, carnuba wax, microcrystalline wax, nonionic emulsifying wax, white wax, and yellow wax.

The coating can be a mixture of ingredients that slow and control drug release, can be applied in quantities not previously achieved, the drug may be in the substrate or in the coating, and the coated beads can be further formulated into chewable controlled release tablets.

In the process of the invention, the coating material is melted such that the molten coating material, when it is combined with the substrate, is at a temperature that is slightly greater than the melting point of the coating material. Preferably, the molten coating material is combined with the substrate at a temperature of 1-30° C., more preferably 5-25° C., and most preferably 10-20° C. or 5-15° C. above the melting point of the coating material. The coating material can be melted at a temperature much higher the melting point if desired but it is generally preferred that the molten coating material is only slightly above its melting point when the substrate is combined with the molten coating material. If a pharmaceutical agent, such as an antigen, is dissolved or dispersed in the molten coating material, preferably the temperature should be at a level such that the pharmaceutical agent is not destroyed. One advantage of the method of the invention and the coated substrates made therefrom, is that the integrity of the pharmaceutical agent can be maintained during processing because the molten coating material is used at a temperature which is only slightly above its melting point and thus, at a temperature that will not destroy the pharmaceutical agent. In addition, the time the pharmaceutical agent must remain at an elevated temperature in the molten coating material is greatly reduced (relative to spray melt coating in a fluid bed where application rates of molten coating must be limited to avoid substrate agglomeration) because the invention allows very rapid transfer, including rapid pouring, of all molten material onto substrate.

The melt coating (the molten coating material) on the substrate of the present invention may comprise one or more coating layers made from the coating material. With regard to the multiple layers of the melt coating material, however, the single-layered coated substrate need not be preheated in each subsequent molten coating application step of the process. Preferably, the coated substrate comprises 1 to 10 layers of melt coating, and more preferably 1-3 or 1-2 layers of melt coating material and most preferably one layer.

The same or different molten coating material can be applied in subsequent applications steps and thus the resulting coated substrate can have multiple layers of the same or different coating material.

The molten coating material of the invention contains little or no solvent. Conventional polymer film coating methods provide a polymer that is dissolved or dispersed in a large volume of solvent and the solvent is driven off leaving a polymer film on the substrate. Typical organic solvents such as alcohol, acetone, dimethyl chloride or the like may denature proteins, require explosion proof equipment, and require special expensive solvent recovery equipment. Current aqueous dispersions of coatings such as ethylcellulose and methyl methacrylate typically contain 70% or more water and about 6-25% polymer by weight of the polymer solution or dispersion, and usually contain plasticizers such as triethyl citrate and/or dibutyl sebacate. Many, such as those used for enteric coatings, are very acidic. The acid and/or the water often promotes degradation of pharmaceuticals. Shear forces that occur during pumping and spraying can be especially damaging to protein compounds, and the current invention can avoid all spraying shear forces. In contrast, the coating method of the present invention does not rely on solvents but rather heat to melt the coating material for application to a substrate. Preferably the coating material contains less than 10% solvent, more preferably 0-5% solvent, still more preferably 0-3%, 0-2% or 0-1% solvent, and most preferably, no solvent or almost no solvent. The meaning of solvent is well understood but also includes, in the context of this invention, a characteristic of being more than 50%, and usually more than 90% removed by evaporation during the coating process and does not necessarily require that the solvent dissolve the coating material. Water, for example, is considered a solvent in aqueous based spray film coating of ethylcellulose in a fluid bed method because the water is evaporated away. In this case, the ethylcellulose is a microdispersion in the water but is not dissolved in the water (solvent). The molten coating material congeals while it cools and thus adheres to the substrate, and does not necessarily adhere due to solvent evaporation. Non-limiting examples of solvents that can be included in small amounts in the coating material of the invention can include water, plasticizers and/or organic solvents such as alcohol, acetone, dimethyl chloride or the like in amounts that do not promote active ingredient (e.g., antigen or pharmaceutical agent) degradation.

The level of coating material in a single coating layer may be greater than 5.5% to 5.6%, 5.7%, 5.8%, 5.9%, or 6% and preferably from about 6 to about 100% and more preferably about 6% to about 60% or about 10% to about 50% of the weight of the coated substrate. The level of coating material in empty capsules may be even greater, for example, up to 500% of the weight of the coating material or more. For multiple layers, the level of coating material may be greater than 5.5% and more preferably greater than 6%, and even more preferably greater than 8% and even more preferably from about 6% to about 2000% and most preferably from approximately 6% to 100%, 8% to 50%, or 10% to 40% of the total weight of the coated substrate, for example, beads, filled capsules or partially filled capsules. Coated empty capsules or partially empty capsules may have single or multiple layers of coating material from approximately 100% to 2000%, 100% to 1500%, 100% to 1000%, and 100% to 500% of the total weight of the substrate. If empty capsules are used as the substrates, the level of coating material can be up to 2000% of the weight of the capsule (as shown in Example 10) or more, but the level of coating material on a bead, in contrast, is typically only up to 75%-100% of the weight of the bead.

Using the method of the present invention, coating material having a greater viscosity can be used without causing agglomeration in comparison to conventional methods. Any suitable coating material that does not cause agglomeration in the method can be used in the process of the invention. The coating material preferably has a molten viscosity of from 10 to 1000 centipoise or greater, but coating materials having molten viscosities of greater than 300, for example from 300 to 1000 cps can be used.

After a molten coating material is applied to a substrate, the coated substrate can then be further coated with a controlled release coating. This additional coating may be applied using known methods such as a spray film coating with a polymer coating agent, for example, by using a fluidized bed of coated substrate in a Wurster column. Please see Achanta, A. S., et al., Drug Development and Industrial Pharmacy (1997) 23:441-449; Ayres, J. W., et al., Pharm. Technol. (1990) 14:72-82; U.S. Pat. No. 5,591,433. Controlled release includes timed release, sustained release, delayed release such as by an enteric coating, and all terms which describe a release pattern other than immediate release. Such coatings may be used, for instance, so that an antigen or pharmaceutical agent that is susceptible to degradation is not degraded by gastric acid, if the direct blend melt coating material does not offer such protection, or does not offer sufficient protection. Gelucire offers some protection against gastric fluids as does stearic acid and mixtures thereof. U.S. Pat. No. 6,194,005 describes particular controlled release forms.

A polymer coating agent is a material applied in a solvent as a film to control drug release, and is not applied by melt coating, and may also be referred to herein as a polymeric rate-release or rate-control materials. Polymeric coating agents include, without limitation, diffusional controlled release polymers, erodible polymers, and enteric coating polymers. The polymeric coating material also may be a bioerodible material that bioerodes at a controlled rate. Preferably, the controlled release coating is an enteric coating using an aqueous methyl methacrylic copolymer dispersion, such as Eudragit L30D 55D, and a plastizer, such as triethyl citrate. Polymer coating agents used in this manner include, but are not limited to, polymethacrylates, ethylcellulose, silicone elastomers, ethylene-vinyl acetate, polyethylene, cross-linked polyvinyl pyrrolidone, vinylidene chloride-acrylonitrile copolymer, polypropylene, polyvalent acid or alkali mobile crosslinked polyelectrolytes, polycarboxylic acid, polyesters, polyamides, polyimides, polylactic acid, polyglycolic acid, polyorthoesters, polyortho-carbonates, and the like. Such polymers, and procedures for forming coatings using the polymers, are disclosed in U.S. patents U.S. Pat. Nos. 3,811,444, 3,867,519, 3,888,975, 3,971,367, 3,993,057 and 4,138,344, which are incorporated herein by reference. Such materials can be applied using methods known in the art, such as the methods described in U.S. patents, U.S. Pat. Nos. 3,938,515, 3,948,262, and 4,014,335, which are incorporated herein by reference.

Any of the foregoing polymeric materials may be used alone to form polymeric coatings. Polymeric materials also may be used in combination, i.e., two or more different polymeric materials may be combined. Moreover, the polymeric materials also may be combined with other materials. For example, polymer-coating materials may also contain plasticizers, such as triethylcitrate or dibutyl sebacate, among many others as well known in the art. Polymer coatings may be applied as a aqueous dispersions or in organic solvents.

Although the method of the invention can be used without the use of a pharmaceutical agent, a preferred embodiment includes the presence of a pharmaceutical agent. As used herein, the term “pharmaceutical agent” includes any suitable element, compound, drug or entity, including, but not limited to, e.g., pharmaceutical, therapeutic, pharmacologic, or a biopharmaceutical compound, or a pharmacologically active agent including a nutrient, such as a vitamin, drug, synthetic compound, or chemical compound. A pharmaceutical agent means any therapeutic or diagnostic agent now known or hereinafter discovered. Examples of therapeutics, without limitation, are listed in Urquhart's U.S. Pat. No. 4,649,043, which is incorporated herein by reference. Additional examples are listed in the American Druggist (February, 1995) 21-24, which is incorporated herein by reference.

The terms “antigen” or “immunogen” are broadly used herein to encompass any chemical or biological substance that elicits an immune response when administered to an animal. While an immunogen is frequently a protein, it may also be a nucleic acid, glycoprotein or polysaccharide. For the purpose of the present invention, immunogens include, but are not limited to, the following: an allergen, a killed bacterium or a bacterial component, a killed virus or a viral component, a peptide, a protein fragment, a protein, such as ovalbumin, a glycoprotein, a gene, a gene fragment, a DNA, an RNA, a polysaccharide or lipopolysaccharide and any combinations of these substances. Examples of allergens include allergenic proteins and digested fragments thereof such as pollen allergens from ragweed, such as ragweed pollen extract, rye, June grass, orchard grass, sweet vernal grass, red top grass, timothy grass, yellow dock, wheat, corn, sagebrush, blue grass, California annual grass, pigweed, Bermuda grass, Russian thistle, mountain cedar, oak, box elder, sycamore, maple, elm and so on, dust, mites, bee and other insect venoms, food allergens, animal dander, animal hair, such as cat hair, microbial vaccines which in turn include viral, bacterial, protozoal, nematode and helminthic vaccines and their various components such as surface antigens, including vaccines which contain glycoproteins or proteins, protein fragments, genes or gene fragments prepared from, for example, Staphylococcus auretis, Streptococcus pyogenes, Streptococcus pneumoniae, Neisseria meningitidis, Neisseria gonorrhoeae, Salmonellae species, Shigellae species, Escherichia coli, Klebsiellae species, Proteus species, Vibrio cholerae, Helicobacterpylori, Pseudomonas aeruginosa, Haemophilus influenzae, Bordetella pertussis, Mycobacterium tuberculosis, Legionella pneumophila, Treponema pallidum, and Chlamydiae species, tetanus toxoid, diphtheria toxoid, influenza viruses, adenoviruses, paramyxoviruses, rubella viruses, polioviruses, hepatitis viruses, herpesviruses, rabies viruses, human immunodeficiency viruses, and papilloma viruses, in addition to protozoal parasites such as Toxoplasma gondii, Pneumocystis carinii, Giardia lamblia, Trichomonas vaginalis, Isospora beeli, Balantidium coli, Blastocystis hominis, and the various species of Entamoeba, Amebae, Plasmodium, Leishmania, Trypanosoma, Babesia, Cryptosporidium, Sarcocystis, and Cyclospora, as well as nematodes and helminths of the various species of trematodes, flukes, cestodes and visceral larvae.

An antigen that can be included in the coated substrate of the invention can be thermally stable or thermally unstable. Preferably, the antigen is a heat sensitive antigen that is not degraded during the application of the molten coating material. One of the advantages of the method of the present invention is that it is possible for the molten coating material to be combined with the substrate and/or antigen at such a low temperature that the antigen (or the pharmaceutical agent) is not degraded. Furthermore, the length of time a heat sensitive antigen or agent is exposed to the heat of a coating material during method of the invention is much less than conventional methods, such as spray melt coating. In a preferred embodiment of the invention, the coating material is made molten and then cooled to near the congealing point before the substrate and/or agent is added, and thus, the substrate and/or agent can be added when temperature of the molten coating material is at an acceptable level such that the heat does not destroy the agent. Conventional methods, therefore, are not suitable for heat-sensitive antigens or agents where the antigen or agent is mixed with molten coating material.

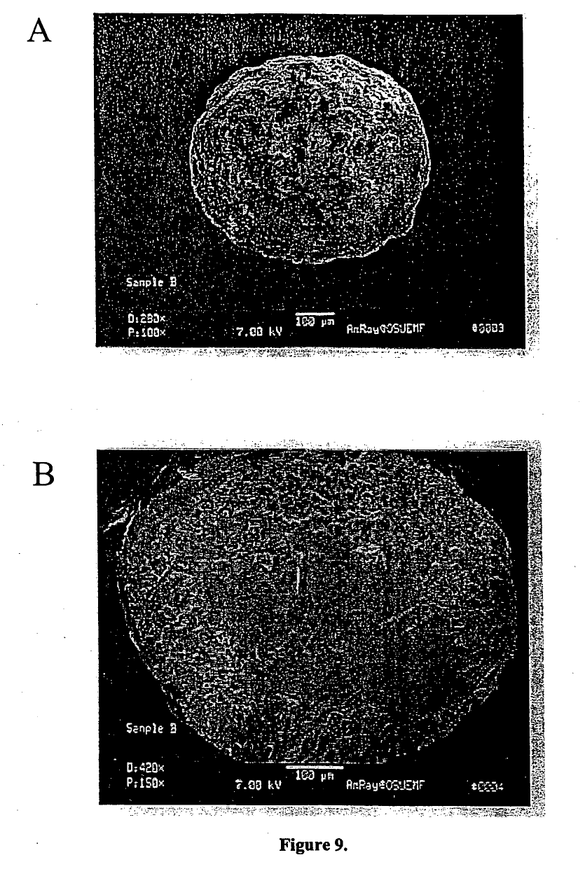

In one embodiment, the pharmaceutical agent or antigen may be found in one or more of the following places in the substrate: within the substrate, on the surface of the substrate, or in the coating material, and most preferably in the coating material. Preferably, the antigen or pharmaceutical agent is uniformly dispersed in the coating material of the coated substrate. Further, Example 7 shows that Gelucire coated beads of the invention are uniform and have a uniform surface, as seen in FIGS. 9-11. It is expected that coating material other than Gelucire will produce uniformly coated substrate because the process revealed is now shown to do so. The results from Example 4 indicate that the coating material is surprisingly uniformly dispersed on the solid support and thus, if the antigen or pharmaceutical agent is found in the coating material, it is expected that it, too, will be uniformly dispersed on the substrate, which has now been shown with respect to ragweed pollen extract and ovalbumin.

In the embodiment where the substrate is a capsule, the pharmaceutical agent or antigen is found in the coating material. In another embodiment, the pharmaceutical agent or antigen is found in the capsule. In a preferred embodiment of the capsule, the pharmaceutical agent or antigen is in the capsule and the capsule is partially full.

Dredan et al., Acta Pharmaceutica Hungarica (1999 September) 69 (4), 176-80 (Abstract) describes a matrix type sample where core material was mixed into the molten mass of thermosoftening natural coating material but does not disclose the method of the invention. In contrast, the present invention is directed to individually coated discrete substrates. However, if for example, a drug forms a matrix with the substrate, which is then coated, these types of matrices as drug/substrate combinations can be used to form discrete coated particles. In addition, if a drug forms a matrix with the melt coating material, then this matrix can now be applied on a substrate and the substrate will form discrete coated particles.

In one embodiment of the invention, one or more adjuvants may be added to the antigen or immunogen before it is added to the coating material or substrate, or the adjuvant may be added directly into the coating material, or the adjuvant may be contained within the substrate or coated onto the substrate before or after the antigens or immunogens are coated. The term “adjuvant,” as used herein, refers to any biological or chemical substance which, when administered with an antigen or an immunogen, enhances the immune response against the immunogen, for example, by either concentrating the antigen or immunogen at a site where lymphocytes are exposed to the antigen or immunogen, or by inducing cytokines which regulate lymphocyte finction. The adjuvant may be either a biological compound, a chemical compound that is therapeutically acceptable, or a combination of a biological and chemical compound. Examples of chemical adjuvants are water dispersible inorganic salts such as aluminum sulfate, aluminum hydroxide (alum) and aluminum phosphate. Examples of biological adjuvants are endogenous cytokines such as granulocyte-macrophage colony-stimulating factor (GM-CSF), tumor necrosis factor-α (TNF-α), interleukin-2 (IL-2), interleukin-4 (IL-4), interleukin-12 (IL-12) and γ-interferon (IFNγ), microorganisms such as BCG (bacille Calmette-Guerin), Corynebacterium parvum, and Bordetella pertussis, bacterial endotoxins such as cholera toxin B (CTB) or heat-labile toxin from E. coli (LT), lipopolysaccharide (LPS), and muramyldipeptide (N-acetyl-muramyl-L-alanyl-D-isoglutamine (MDPI). Commercially available adjuvants such as DETOX˜PC® may also be used.

Another aspect of the invention includes a method to induce an immune response in a subject against an allergic reaction comprising administering to a subject in need thereof an effective amount of the coated substrate comprising an allergen. Preferably the method relates to an allergen, preferably ragweed pollen extract, but can also include grasses, weeds, trees, flowers, molds, epidermals, dust mites, insects and insect venoms, foods and latex allergens.

The term “subject,” as used herein, includes humans and animals, including mammals and non-mammals.

The application of the coating can be performed in any suitable vessel providing adequate mixing to both insure coating and prevent agglomeration. Typical air supported fluidized beds, such as Wurster column spray coating fluid bed systems, for example, are not suitable vessels as cited above. Some non-limiting examples include a temperature controlled vat or granulator that preferably contains or is associated with a mechanical mixing apparatus, a scraper, blending blades and/or a propeller mixing apparatus. Ointment formulation vessels with mixing and scraping capability are suitable. An apparatus for dispensing molten coating can include as non-limiting examples a simple pouring spout, or ladle, or pump system that delivers the material through an orifice with minimal or no shear. Spray delivery is possible but not preferable for reasons cited above. Advantageously, the present method can achieve the same or better results using fewer steps than known spray melt coatings or spray film coatings processes.

In one embodiment, the coating material is melted in a different chamber than the chamber in which the molten coating material is applied, and in another embodiment, the same chamber is used to both melt the coating material and apply the molten coating material to the substrate.

In a preferred embodiment, the coated substrates are stirred during the application of the molten coating material and/or thereafter while the coating material congeals and forms a coated substrate, in order to prevent agglomeration. Preferably little or no agglomeration is present. In one further aspect of the inventive method, any agglomerates that may form after the molten coating material is applied to the substrate may be removed, for example, by screening through sieves, before any subsequent coating layers are applied to the coated substrate. Agglomerates may also be separated if they are first broken up and then any remaining agglomerates can be separated. This is in contrast to melt-coating by fluid bed spraying methods, agglomeration in a fluidized bed usually results in seizing or possibly a complete loss of the batch of coated substrates due to the weight/mass of the agglomerates that may form that prevent the particles in the fluidized bed from remaining separated by the flow of air. For example, in a Wurster column, an excessive amount of coating may cause the bottom of the funnel to plug up, thereby resulting in loss of the batch.

The following examples are intended to illustrate but not to limit the invention.

### EXAMPLES

Nonpareils (Sugar Spheres NF) were purchased from CHR Hansen, Inc. (Wahwah, N.J.). Stearic acid was purchased from Aldrich Chemical Company, Inc. (Milwaukee, Wis.). Ovalbumin (crude, dried egg white) was purchased from Sigma Chemical Company (St. Louis, Mo.). Ragweed pollen extract (RPE, lot# AF00200T) was purchased from Hollister-Stier Laboratories LLC (Spokane, Wash.). RPE is a lyophilized powder that consists of numerous proteins and carbohydrates. Eudragit® L30D-55 was purchased from Rohm America Inc. (Somerset, Mass.) as enteric coating material is an aqueous methyl methacrylic copolymer dispersion. Film formed from Eudragit L30D 55S remains intact in gastric fluid (pH<5) and dissolves in intestinal fluid (pH>5). Polyethylene glycol 1450, 4600, and 8000 were purchased from Union Carbide Corporation (Danbury, Conn.), and polyethylene glycol 3350 was purchased from Spectrum Chemical Mfg. Corp. (Gardena, Calif.). Gelucire® 50/13 pellets were purchased from Gettefosse Corporation (Westwood, N.J.).

### Example 1

### Process for Producing Coated Ragweed Pollen or Ovalbumin Particles

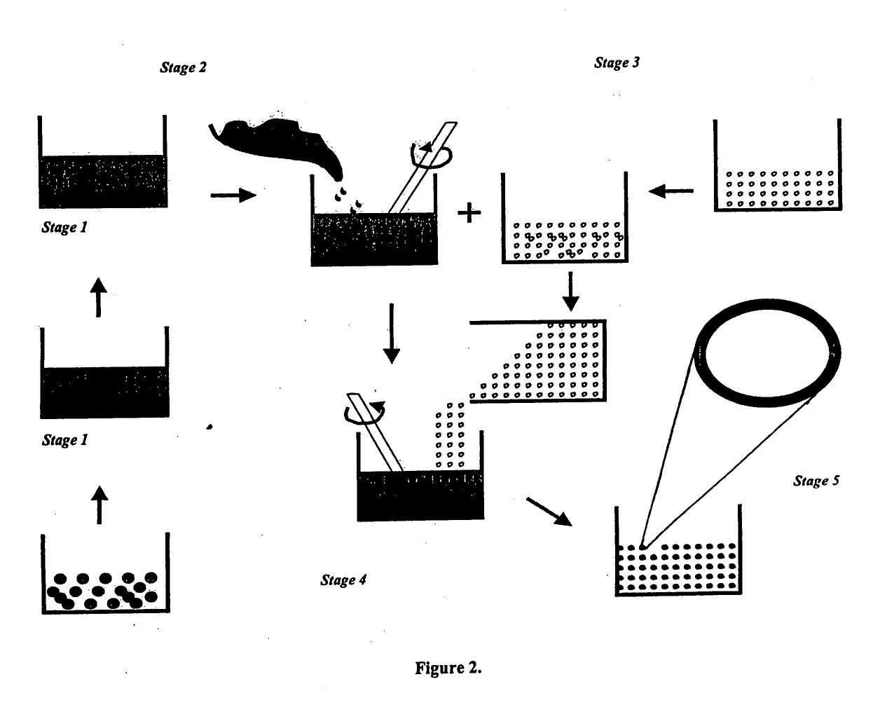

Two batches of Gelucire® 50/13 hot-melt coated particles were produced with Ragweed pollen extract (RPE) and ovalbumin as model active compounds. The hot-melt formulations for these batches are described in Table 1 and Table 2, respectively. The preparation process for the hot-melt coating with RPE is described below. The same process was used for producing ovalbumin particles. The following method is shown schematically in FIG. 2.

About 85 g of nonpareils (30-35 mesh) were weighed out into a beaker, which was. covered and was then placed into a water bath preset at 43° C. During the nonpareil preheating period, occasional tumbling of the beaker was applied to generate uniform temperature distribution among the nonpareils. About 9.5 g of Gelucire® 50/13 were weighed out into a beaker, which was placed into an oven preset at 70-75° C. Upon complete Gelucire® 50/13 melting (10-15 minutes after being placed into oven), the molten Gelucire® was cooled to 43° C. About 0.9 g of RPE immediately was transferred into the molten Gelucire®. A spatula was used to disperse the RPE into the molten Gelucire® by manual mixing and blending. The RPE dispersion (a matrix of RPE in Gelucire) was carried out in a water bath at 43° C. The nonpareils, equilibrated to 43° C., were poured into the beaker containing RPE and molten Gelucire®. This rapid and essentially immediate transfer of all melt coating to the beads cannot be successful in a spray melt coated fluid bed process. Manual mixing/blending was applied to the combination to spread and distribute evenly the hot-melt onto the nonpareil surface. The temperature was maintained at 43° C. for the entire blending process (about 5-10 minutes). Upon the completion of blending, the beaker containing coated nonpareils was removed from the water bath and placed under ambient conditions for congealment and cooling. Occasional stirring was applied to the congealing beads to prevent agglomeration.

Once the beads had reached ambient temperature, the RPE-dispersed molten Gelucire® coated beads were screened through the U.S. Standard Testing Sieve No. 18 to remove any agglomerates. The final hot-melt coated nonpareils, which were highly free-flowing, were subsequently enteric coated.

The enteric coating was performed using a feasibility scale, Fluid Air bed processor with a Wurster Bottom spray insert (Fluid Air, Aurora, Ill.). The enteric coating solution (16% solids), containing Eudragit L30D 55D (34.27 g) and triethyl citrate (3.42 g) as plasticizer, was sprayed onto the Gelucire® 50/13 hot-melt coated nonpareils (70 grams) containing RPE. The coating solution was pumped to the atomizer by a peristaltic pump (MasterFlex® L/S™ Model 77200-50, Cole Parmer Instrument Company, Vernon Hills, Ill.) at a rate of 0.5-1.0 ml/min through a tygon tube (MasterFlex® Tygon® L/S® 14 LFL Tubing, Cole Parmer Instrument Company, Vernon Hills, Ill.), using a spray riozzle having orifice diameter of 0.5 mm (Fluid Air, Aurora, Ill.). The inlet temperature was set at 25-26° C. throughout the entire coating process. At the end of the coating procedure, the beads were dried in the coating chamber for about 60 minutes at 45° C. The atomization air pressure was lowered to 2-3 psi during drying. The processing conditions are summarized in Table 3.

TABLE 1
Gelucire ® 50/13 hot-melt coating
formulations with ovalbumin

| | | | | Percent |
|---|---|---|---|---|
| | Nonpareils | | | Gelucire ® |
| | (30-35 | Gelucire ® | | 50/13 of |
| Hot melt | mesh) | 50/13 | Ovalbumin | Nonpareils |
| Formulation | (g) | (g) | (g) | (%) |
| Hot Melt Coating | 90 | 10 | 1 | 11.11 |
| containing | | | | |
| Ovalbumin | | | | |

TABLE 2
Gelucire ® 50/13 hot-melt coating
formulation with RPE

| | Nonpareils | | | Gelucire ® 50/13 as |
|---|---|---|---|---|
| Hot melt | (30-35 mesh) | Gelucire ® | RPE | Percent of |
| Formulation | (g) | 50/13 (g) | (g) | Nonpareils (%) |
| Hot melt | 85 | 9.5 | 0.9 | 11.18 |
| coating | | | | |
| containing | | | | |
| RPE | | | | |

TABLE 3
Processing conditions imposed for enteric coating of
Gelucire ® 50/13 hot-melt coated
nonpareils containing RPE.

| Parameter | Setting |
|---|---|
| a. Batch size | 108 grams |
| Nozzle size | 0.5 mm |
| Wurster insert | Bottom spray |
| Spraying rate | 0.5-1.0 ml/min |
| Fluidization air flow (SCFM) | 12 |
| Inlet temperature | 25-26° C. |
| Outlet temperature | 25-26° C. |
| Product temperature | 25-26° C. |
| Atomization air flow | 6-7 psi |
| Filter cleaning pressure | 20 psi |

### Example 2

### Increasing Coating Amount on Single Layer Using Direct Blending Hot-Coat Method

In the application of a melt coat to a bead core by conventional processing methods (e.g., solid dispersion hot melt coating in an air fluidized bed) the thickness of melt coat layer is limited by processing difficulties. In order to show that the inventive method is capable of producing single-layer, hot-melt coatings at a greater level than conventional methods when coupled with rapid application of the hot melt coating to substrate, the direct blending hot-melt coating method of the invention was used using Gelucire® 50/13 as the coating agent. The increased hot-melt coating level in a single layer was defmed as the percent weight gain in Gelucire® attained. The process described in Example 1 was followed, however, no active model compound or enteric coating was applied. The Gelucire® levels chosen for this determination were a 20% and 30% in weight gain as indicated in Table 4 along with the coating results.

TABLE 4
Coating formulation and results for determining the maximum Gelucire ® coating
level in a single layer under certain conditions.

| | Number | | Theoretical | | |
|---|---|---|---|---|---|
| Nonpareils | of | | % weight | Actual % weight | Agglomerates: |
| (30-35 | coating | Gelucire ® | gain from | gain from | significant/ |
| mesh) (g) | layers | 50/13 (g) | Gelucire | Gelucire | non-significant |
| 45 | 1 | 9 | 20 | 17.11 | Non-significant |
| 45 | 1 | 13.5 | 30 | 24.22 | Slightly |
| | | | | | significant |

The results indicate that the increased achievable percent weight gain from Gelucire® following the process of the invention that does not result in significant agglomeration is at least between 20 and 30% for a single coating layer under the conditions indicated, which includes rapid application of the hot melt coating to substrate. This experiment was done in a beaker with a stirring rod.

It is expected that the achievable percent weight gain can be optimized such that weight gains up to 100% or more may be made.

### Example 3

### Increasing Coating Amount on Each Single Layer for Multiple Layer Coating

Multiple layers of coating with Gelucire® 50/13 using the process of the invention on nonpareils was conducted in order to obtain weight gains in excess of 20 percent that was achieved in a single layer as shown in Example 1. The hot-melt coating by direct blending procedure described in Example 1 was followed for the multiple layer coating using Gelucire® 50/13. However, the nonpareils were only preheated to 43° C. for the first layer of hot-melt coating. Subsequent layers of hot-melt coating were applied without preheating the already-coated nonpareils before mixing/blending them with the molten Gelucire®. Two multiple coating procedures are described. In the first demonstration, a five-layered hot-melt coating, with an intended weight gain after each layering equal to 20% of the starting nonpareil weight was produced (Table 5). In a second demonstration, a three-layered hot-melt coating with an intended Gelucire® weight gain in each layering equal to 30% of the starting nonpareil weight was produced (Table 6). The results of this study indicate that a weight gain of at least 79.5% is achievable on nonpareil beads through a multiple layer hot-melt coating process of the invention under these conditions. Kennedy, supra, teaches that the maximum feasible hot-melt coating level using even his improved solid dispersion hot-melt fluid bed coating method in a substrate bed fluidized on air with a Wurster bottom spray insert can only be varied from 2.5 percent to 5.5 percent depending on different substrate sizes. It is expected that the condition under which the multiple layered coated substrate is formed can be optimized in order to achieve a weight gain of at least 300%.

TABLE 5
Multiple layer hot-melt coating: Five-layered hot-melt coating system.

| | | | Achieved | | |
|---|---|---|---|---|---|
| | | Intended total | total | Gelucire | Gelucire |
| | | amount | amount | loss (g) at | loss (%) at |
| Layer # | Initial amounts (g) | (g) | (g) | each layer | each layer¹ |
| 1 | Nonpareils: 45 | 54 | 52.7 | 1.3 | 14.44 |
| | Gelucire: 9 | | | | |
| 2 | Coated nonpareils: | 61.7 | 60 | 1.7 | 18.88 |
| | 52.7 | | | | |
| | Gelucire: 9 | | | | |
| 3 | Coated nonpareils: 60 | 69 | 67.1 | 1.9 | 21.11 |
| | Gelucire: 9 | | | | |
| 4 | Coated nonpareils: | 76.1 | 74 | 2 | 22.22 |
| | 67.1 | | | | |
| | Gelucire: 9 | | | | |
| 5 | Coated nonpareils: 74 | 83 | 80.8 | 2.2 | 24.44 |
| | Gelucire: 9 | | | | |

¹Assume that the loss was only attributed to Gelucire, not the nonpareils.

TABLE 6
Multiple layer hot-melt coating: Three-layered hot-melt coating system.

| | | | Achieved | | |
|---|---|---|---|---|---|
| | | Intended total | total | Gelucire | Gelucire |
| | | amount | amount | loss (g) at | loss (%) at |
| Layer # | Initial amounts (g) | (g) | (g) | each layer | each layer¹ |
| 1 | Nonpareils: 45 | 58.5 | 55.9 | 2.6 | 19.26 |
| | Gelucire: 13.5 | | | | |
| 2 | Coated nonpareils: | 69.4 | 66.5 | 2.9 | 21.48 |
| | 55.9 | | | | |
| | Gelucire: 13.5 | | | | |
| 3 | Coated nonpareils: | 80 | 76.4 | 3.6 | 26.67 |
| | 66.5 | | | | |
| | Gelucire: 13.5 | | | | |

¹Assume that the loss was only attributed to Gelucire, not the nonpareils.

### Example 4

### Drug Content Uniformity Assessment

Drug content uniformity study was performed on Gelucire® 50/13 hot-melt coated nonpareils containing antigen (RPE or ovalbumin) before application of the enteric coating layer. This assessment was conducted to evaluate the feasibility and robustness of the hot melt coating by direct blending method of the invention. Given the size of the RPE batch prepared (about 100 grams), twelve random 15 milligram samples of Gelucire coated nonpareils were weighed and placed into 0.5-milliliter microcentrifuge tubes (Fisher Scientific, Pittsburgh, Pa.) for sample preparation. Three hundred microliters of deionized water were employed as dissolution medium for each 15-microgram sample. The microcentrifuge tubes were vortexed for 1-2 minutes for complete antigen dissolution into the medium. The tubes were then centrifuged for about 10 minutes to obtain a clear solution with insoluble excipients precipitated on the bottom. In a randomized order from each of the twelve centrifuge tubes, an aliquot of 10 microliters of clear solution was pipetted into separate cell wells on a NUNC Immuno™ plate (Nalge NUNC International, Denmark). The samples were analyzed for protein content by bicinchoninic acid (BCA) protein assay.

The BCA protein assay is a detergent-compatible formulation based on bicinchoninic acid for calorimetric detection and quantification of total protein. BCA protein assay reagent is prepared by combining one part of BCA reagent B with fifty parts of BCA reagent A (Product No. 23224 and 23223, respectively, Pierce, Rockford, Ill.). The assay reagent is stable for 24 hours post preparation when stored in a closed container at room temperature. Samples (10 μl) collected from drug release studies or drug uniformity studies were developed by adding 200 microliter BCA protein assay reagent into each of the cell wells containing the samples on a NUNC Immuno™ plate (Nalge NUNC International, Denmark). The NUNC Immuno™ plate was incubated at 37° C. for about 1 hour before analysis by microplate reader (Multiskan MCC, MTX lab system, Inc., Vienna, Va.) at a wavelength of 540 nm for protein concentrations.

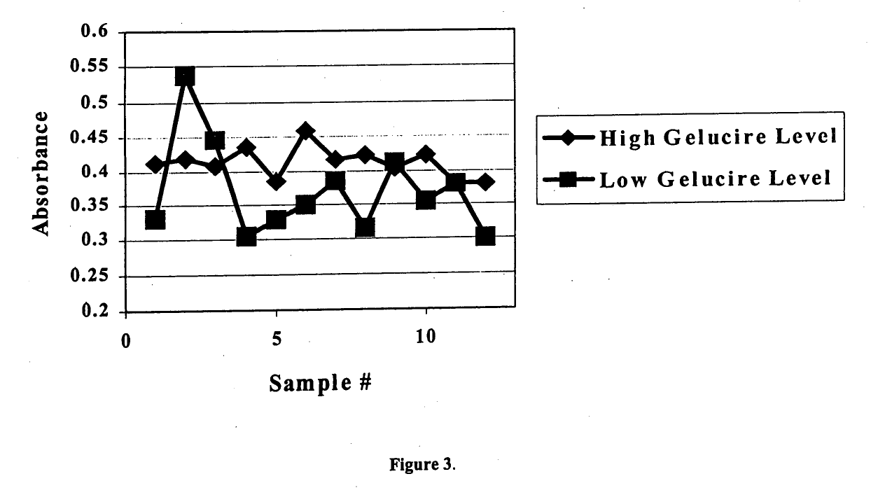

Desirable hot-melt coating amounts were approximated with two coating formulations of lower and higher weight gains of Geluicre® 50/13. The two coating formulations are given in Table 7. Ovalbumin was utilized as model compound. As indicated in FIG. 3, ovalbumin content distribution from the lower Gelucire coating (formulation A, Table 7) was less consistent than that from the higher Gelucire coating (formulation B, Table 7). The relative standard deviations of samples from the two different batches were estimated to be 18.53% and 5.68%, respectively for formulations A and B in Table 7. According to the USP <905>Uniformity of Dosage Units (The United States Pharmacopoeia and National Formulary, 24^{th} ed., United States Pharmacopeial Convention, Rockville, Md., 2000), “the requirements for dosage uniformity are met if the amount of the active ingredient in each of the 10 dosage units as determined from the relative standard deviation is less than or equal to 6%”. A Gelucire coating level of 11.11% in weight gain produced a relative standard deviation of 5.68%, thus Gelucire coating of about 11% in weight gain may be considered to meet requirements for dosage uniformity for ovalbumin under these conditions. Other coating amounts may be preferred for other drugs. In some cases relative standard deviations of more than 6% may be acceptable. It is surprising that such uniform coats are obtained using the direct blending hot-melt coating method of the invention as one-by-one hand dipping of tablets in relatively low viscosity solvent coatings results in more variable coating weight gain.

TABLE 7
Gelucire ® 50/13 hot-melt coating formulations
for drug content uniformity comparison with
ovalbumin as model protein compound.

| | | | | Percent |
|---|---|---|---|---|
| | Nonpareils | | | Gelucire ® |
| | (30-35 | Gelucire ® | | 50/13 of |
| Hot melt | mesh) | 50/13 | Ovalbumin | Nonpareils |
| Formulation | (g) | (g) | (g) | (%) |
| Hot Melt Coating | 95 | 5 | 1 | 5.26 |
| Formulation A | | | | |
| (Low level) | | | | |
| Hot Melt Coating | 90 | 10 | 1 | 11.11 |
| Formulation B | | | | |
| (High level) | | | | |

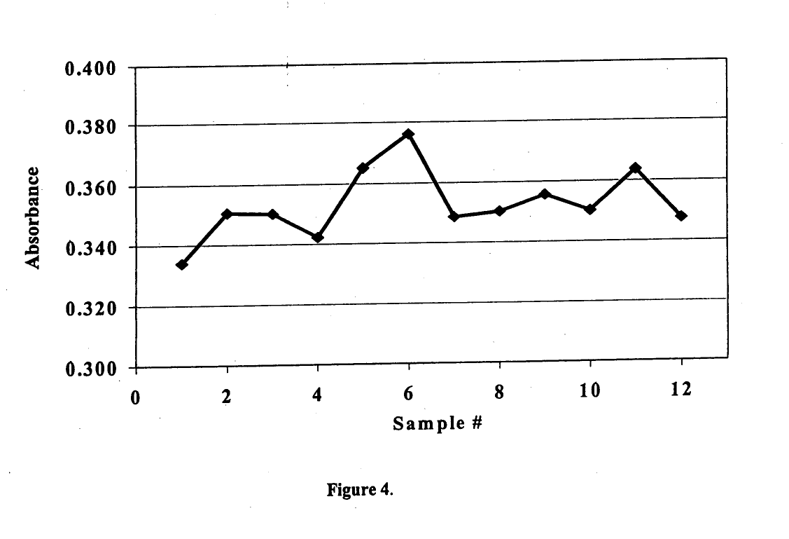

The result for drug content uniformity was found to be reproducible with the direct blending hot-melt coating method of the invention when ovalbumin was replaced by RPE as a model protein compound (FIG. 4). The relative standard deviation was estimated to be about 3.12% for a group of 12 random samples selected from a batch of 95.4 gram coated beads. The Gelucire coating level applied was about 11.18% in weight gain (Table 8).

TABLE 8
Gelucire ® 50/13 hot-melt coating
formulation for drug content uniformity using
RPE as model protein compound

| | Nonpareils | | | Gelucire ® 50/13 |
|---|---|---|---|---|
| Hot melt | (30-35 | Gelucire ® | | as Percent of |
| Formulation | mesh) (g) | 50/13 (g) | RPE (g) | Nonpareils (%) |
| Hot melt coating | 85 | 9.5 | 0.9 | 11.18 |
| containing RPE | | | | |

The acceptable relative standard deviations (5.68% and 3.12% for ovalbumin and RPE, respectively) from the content uniformity testing clearly demonstrate that the direct blending hot-melt coating method of the invention can produce coated substrates containing an antigen or pharmaceutical agent in uniform amounts. As a result of these results with suspensions, drugs that are soluble in direct blending hot-melt coatings are expected to show acceptable content uniformity.

RPE is a protein containing antigen prepared by aqueous extraction of ragweed followed by freeze drying to avoid thermodegradation. This example shows that the RPE protein containing antigen is unexpectedly stable when this heat sensitive antigen is mixed into a molten coating material.

### Example 5

### In Vitro Drug Dissolution Studies

Drug dissolution studies were performed on the enteric-coated Gelucire® 50/13 hot-melt coated nonpareils containing RPE. A modified USP dissolution method for delayed release formulations was employed for the RPE dissolution studies. The drug dissolution study consisted of two separate tests; one test conducted in simulated gastric fluid (0.1 N HCl) while the other was in simulated intestinal fluid (USP phosphate buffer). Using modifications to the dissolution method, in vitro drug release was carried out in a 15 ml screw cap centrifuge tube (PGC Scientifics, Frederick, Md.), which was placed onto a bench-top rocker (Model 35/35D, National Labnet Company, Woodbridge, N.J.) for simulated intestinal mixing. The amount of motion for the centrifuge tubes was controlled by turning the knob on the bench-top rocker to indicate “5”, which was chosen traditionally in this laboratory for conducting dissolution studies. The modified dissolution process was carried out at room temperature.

Two randomly selected samples, 350 mg each, of enteric-coated RPE beads were selected from a finished batch and placed separately into two screw cap centrifuge tubes followed by addition to each tube of 3.5 ml of simulated gastric fluid and simulated intestinal fluid, respectively. An aliquot of 10 μl sample was taken at 30, 60, 90, and 120 minutes for RPE release in the simulated gastric fluid and at 15, 30, and 45 minutes for RPE release in the simulated intestinal fluid. The 10 μl samples from each fluid were then placed into cell wells of a separate column on a NUNC Immuno™ plate (Nalge NUNC International, Denmark) in ascending order of preset sample times. The samples were analyzed for drug content by bicinchoninic acid (BCA) protein assay described above.

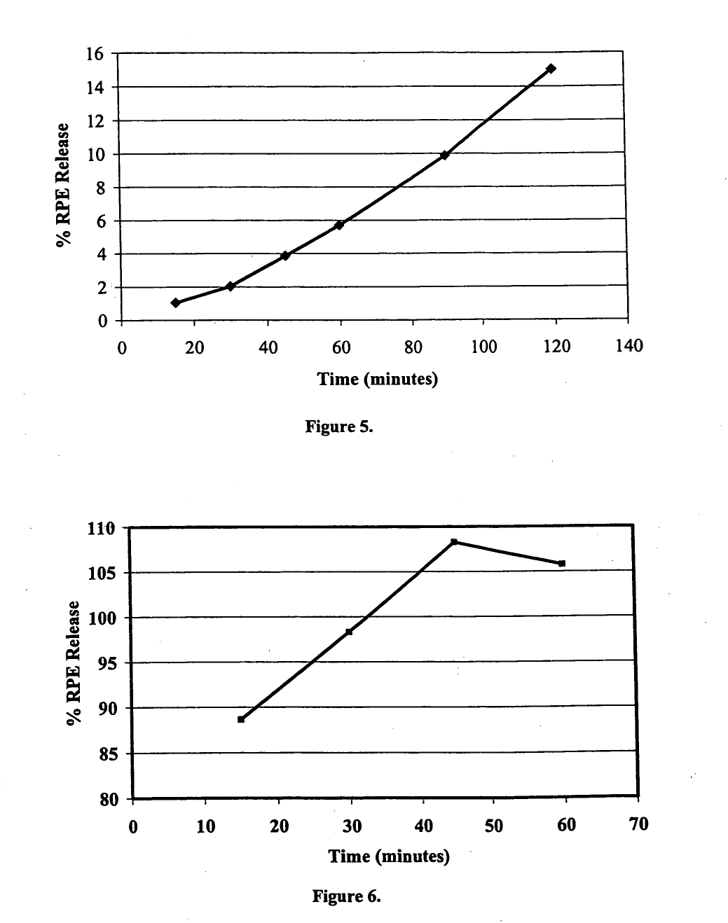

The Gelucire hot-melt coated beads containing RPE (Table 7) were spray-coated with Eudragit L30D 55S. The enteric coating layer obtained was about 26.64% (w/w). In vitro drug dissolution studies were conducted on the enteric-coated beads in both simulated gastric fluid (SGF) and intestinal fluid (SIF). The RPE release profiles in SGF and SIF are shown in FIG. 5 and FIG. 6, respectively. The percent RPE released at the end of two hours was estimated to be about 15% in SGF. Since the solubility of Eudragit L30D 55S film is pH dependent and can only start to dissolve at pH 5.0 or higher, the observed RPE concentration in SGF thus indicated that RPE was released mostly by diffusion through the enteric coating layer. This implied that a thicker enteric coating layer would be preferred to further retard drug release in the stomach given that the intended RPE release site is the small intestine.

RPE release was approximately complete after 45 minutes in the SIF (FIG. 6), which was indicative that Eudragit L30D 55S enteric-coating layer was dissolved fully and that Gelucire hot-melt coating layer (based on coating formulation described in Table 7) was also dispersed completely. Gelucire® 50/13 has been employed for controlled drug release formulations. The lack of extensive sustained drug release was most likely the result of the relatively thin Gelucire coating amount applied when compared to the amount necessary for generating a significant controlled release effect, which was not included in this example but Examples 3 and 4 showing that much more coating can be applied teach that the direct blending hot-melt coating method can be used for sustained action formulations especially when coupled with Example 10 showing use of a different coating mixture.

Protein Standard Curve Preparation

The drug standard curve was prepared for drug dissolution studies because the drug content uniformity study did not investigate drug release as a finction of time. Since drug dissolution is conducted in both the simulated gastric fluid and the simulated intestinal fluid, two protein standard curves are generated, one in each media.

About 350 mg of enteric-coated RPE beads are ground using a mortar and pestle. The powder is transferred into a 15 ml screw cap centrifuge tube (PGC Scientifics, Frederick, Md.). A 3.5 ml volume of dissolution medium is pippetted into the centrifuge tube, which is then vortexed for about 2-3 minutes for complete RPE dissolution followed by a 2-3 hour static sedimentation of insoluble particulates. The sedimentation process results in an RPE solution that is used as the stock solution for the standard curve preparation (representing 100% release). The standard curve dilutions are performed according to Table 9 by transferring the specified volume of stock solution and the dissolution medium to clean, glass disposable test tubes.

TABLE 9
Standard curve dilution method

| Standard Level (%) | Stock Solution (μl) | Dissolution Media* (μl) |
|---|---|---|
| 100 | 400 | 0 |
| 80 | 400 | 100 |
| 60 | 300 | 200 |
| 40 | 200 | 300 |
| 20 | 100 | 400 |
| 10 | 100 | 900 |
| 5 | 100 | 1900 |

*The dissolution media include both the 0.1N HCl solution (pH 1.29) and the USP Buffer (pH 6.8).

The glass disposable test tubes were then vortexed for about 30 seconds for uniform mixing of the stock solution and the dissolution medium. For each of the standard levels, a 10 μl sample was pippetted into one cell well of a column on a NUNC Immuno™ plate (Nalge NUNC International, Denmark) in ascending concentration. Normally, the same NUNC Immuno™ plate was utilized for both the standard curve and the drug dissolution samples of which the standar curve was used to quantify the drug levels. Bicinchoninic acid (BCA) protein assay was then performed on the Immuno™ plate according to the BCA protein assay procedure described above.

### Example 5A

Hot melt coating by direct blending beads containing Verapamil hydrochloride as a model drug were prepared. Ingredients are shown in the table below.

| | Sugar Beads | | |
|---|---|---|---|
| | 30-35 mesh | Melt Coating | Verapamil |
| Formulation | Paulaur Corp. | Material | Hydrochloride |
| Number 1 | 100 grams | Gelucire 50/13 | 7 grams |
| | | (15 grams) | |
| Number 2 | 100 grams | PEG 4000 (15 grams) | 7 grams |
| Number 3 | 100 grams | Carnauba wax | 7 grams |
| | | (15 grams) | |
| Number 4 | 100 grams | Synchrowax HGL-C | 7 grams |
| | | (15 grams) | |
| Number 5 | 100 grams | Synchrowax BB4 | 7 grams |
| | | (15 grams) | |

Gelucire 53/13-received from Gateffosse Corporation;
PEG 4000 received from Dow Corporation;
Synchrowax HGL-C (C18-C36 acid triglycerides) received from Croda Inc.;
Synchrowax BB4 (synthetic beeswax) received from Croda Inc.

The melt coating material was melted in a beaker at a temperature about 10 degrees centigrade above the melting point and the drug dispersed in the molten material with stirring using a spatula. In a separate container, the beads were warmed to the same temperature as the molten material and then the beads were added directly into the molten melt-coating/drug mixture with stirring. Stirring was continued while the mixture was allowed to cool to room temperature and then the melt-coated product was passed through a 25 mesh sieve. In some cases this process was repeated to apply multiple layers of material to obtain the final product as desired. Dissolution studies were conducted on the coated beads of formulations 1-5 in a United States Pharmacopia apparatus II with paddle stirring at 50 RPM in USP simulated gastric fluid without enzymes at 37.5 degrees centigrade. Results are shown in the table below.

| | % drug | % drug | % drug | % drug | % drug |
|---|---|---|---|---|---|
| Time | released | released | released | released | released |
| (Hours) | Formulation 1 | Formulation 2 | Formulation 3 | Formulation 4 | Formulation 5 |
| 0 | 0 | 0 | 0 | 0 | 0 |
| 0.25 | 97.1 | 97.1 | 20.9 | 16.3 | 14.0 |
| 0.5 | 99.1 | 99.1 | 39.9 | 26.4 | 23.7 |
| 1 | 99.5 | 99.9 | 42.6 | 41.4 | 33.3 |
| 1.5 | 99.9 | 100 | 56.5 | 53.2 | 51.3 |
| 2 | 100 | 100 | 65.6 | 60.6 | 58.3 |
| 3 | 100 | 100 | 70.6 | 64.6 | 67.9 |
| 4 | 100 | 100 | 80.2 | 68.4 | 73.5 |
| 5 | 100 | 100 | 85.3 | 75.7 | 80.6 |
| 6 | 100 | 100 | 90.2 | 82.6 | 85.7 |
| 7 | 100 | 100 | 93.9 | 87.9 | 89.9 |

In this example more than 20% weight gain of coating was applied onto the pharmaceutical substrate sugar beads. The coating material was 31.8% drug and 68.2% melt coating excipient. Release of drug can be controlled by the melt coating materials as shown in the table above. Drug release was, for example, 99% at 0.5 hours for Formulations 1 and 2 but only 23.7% for Formulation 5 at 0.5 hours. The data show that Formulations 1 and 2 are good immediate release formulations while Formulations 3, 4 and 5 are good sustained release drug formulations made using the new method disclosed herein.

Formulations 1-5 above contains a drug/carrier ratio of 0.47. Two additional formulations were prepared containing Carnuba wax with a drug/carrier ratio of 2.75 (formulation 3A) or 1.0 (Formulation 3B). The melt-coating by direct blending as described above process resulted in a weight gain of 30% for formulation 3A and 22% for formulation 3B. Dissolution results under the above conditions are shown below in comparison to dissolution results for Formulation 3.

| Time | % drug released | % drug released | % drug released |
|---|---|---|---|
| (Hours) | Formulation 3A | Formulation 3B | Formulation 3 |
| 0 | 0 | 0 | 0 |
| 0.25 | 64.8 | 47.7 | 20.9 |
| 0.5 | 87.7 | 56.4 | 39.9 |
| 1 | 97.2 | 67.8 | 42.6 |
| 1.5 | 98.8 | 73.5 | 56.5 |
| 2 | 99.3 | 77.3 | 65.6 |
| 3 | 100 | 83.5 | 70.6 |
| 4 | 100 | 88.7 | 80.2 |
| 5 | 100 | 93.5 | 85.3 |
| 6 | 100 | 97.0 | 90.2 |
| 7 | 100 | 100 | 93.9 |

These data demonstrate that variation of the ratio of drug/carrier is readily programmed in the new process by one skilled in the art to produce a variety of immediate or sustained relase profiles from which to choose.

### Example 6

### Dissolution in Acid and Base of Hot-Melt Filled Capsules

Gelucire® 50/13 hot-melt filled capsule was prepared. First, Gelucire® 50/13 hot-melt was prepared which contained Ragweed Pollen Extract (RPE) as a model compound. The hot-melt was then placed into a gelatin capsule and the hot-melt filled capsule was then capped off. The hot-melt filled capsule was allowed to cool down to room temperature. The hot-melt formulation is given in Table 10. The hot-melt preparation and capsule filling processes are describe below.

About 2.5 g of Gelucire® 50/13 was weighed into a beaker, which was placed into an oven preset at 70-75° C. Upon complete Gelucire® 50/13 melting (10-15 minutes after being placed into oven), the oven was allowed to cool to 43° C. About 0.25 g of RPE was then weighed and immediately transferred into the molten Gelucire®. A spatula was used for RPE dispersion into molten Gelucire® by manual mixing and blending. The RPE dispersion preparation was carried out in a water bath at 43° C.

After obtaining a uniform RPE dispersion, the molten hot-melt was transferred by a spatula into a 1-ml syringe. Capsules of 3CS size were filled with hot-melt by controlling the piston movement down the syringe shaft. The filled capsules were then capped off with the gelatin capsule head and allowed to cool down to room temperature. Process temperature should be kept at 40-45° C. to retain ease of capsule filling with Gelucire® 50/13 hot-melt.

TABLE 10
Gelucire ® 50/13 hot-melt formulation for capsule filling

| Hot melt | Gelucire ® 50/13 | RPE |
|---|---|---|
| Formulation | (g) | (g) |
| Weight | 2.5 | 0.25 |
| Percent (%) | 90.91 | 9.09 |

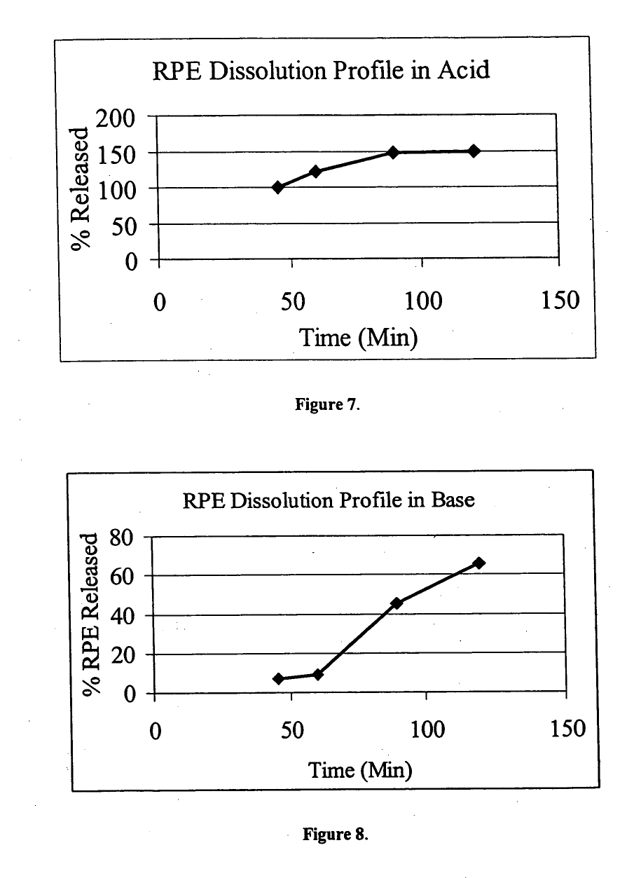

Gelucire 50/13 hot-melt filled capsules were then studied for dissolution in both acid (0.1 N HCl) and base (Buffer pH 6.8). The dissolution profiles are shown in FIG. 7 and FIG. 8 in acid and base, respectively.

### Example 7

### Hot-Melt Coating Layer Morphology Using Scanning Electron Microscopy (SEM)

Gelucire® 50/13 hot-melt coated nonpareils containing RPE, both before and after enteric coating, were examined under a scanning electron microscope (SEM) (Amray 1000A, Amray, Bedford, Mass.) to determine morphological differences in the coating layers. Both the surface (×100) and cross-section SEMs (×150) were examined. To obtain the cross-section SEM view, the coated particles were first cut in half by a surgical blade and then coated with 60:40 gold-palladium alloy before SEM observation.

For Gelucire hot-melt coated (11.11% in weight gain) beads, the scanning electron micrograph (×100) revealed that beads were covered completely by the coating material and that the coating surface was somewhat wavy with occasional small pits or abrasions that become visible with this magnification. (FIG. 9a). The coating also appeared to be waxy and filled with crests and troughs evenly distributed across the surface. These observations are in agreement with the report of Ratsimbazafy, et al. (Bourret, E., et al., J. Pharm. Pharmacol. (1977) 49:852-857) that Gelucire® 50/13 possesses a ribbon-like structure under SEM. The surface irregularity could also be contributed to attrition during the direct blending process in which the adhesion-separation cycle of the coated beads imparted the observed surface appearance.

The cross-section view (×150) of the Gelucire hot-melt coated nonpareils failed to capture any distinctive hot-melt layer thickness (FIG. 9b). This is most likely due to possible hot-melt penetration into nonpareil surface pores coupled with the large nonpareil surface area to the very thin hot-melt coating.

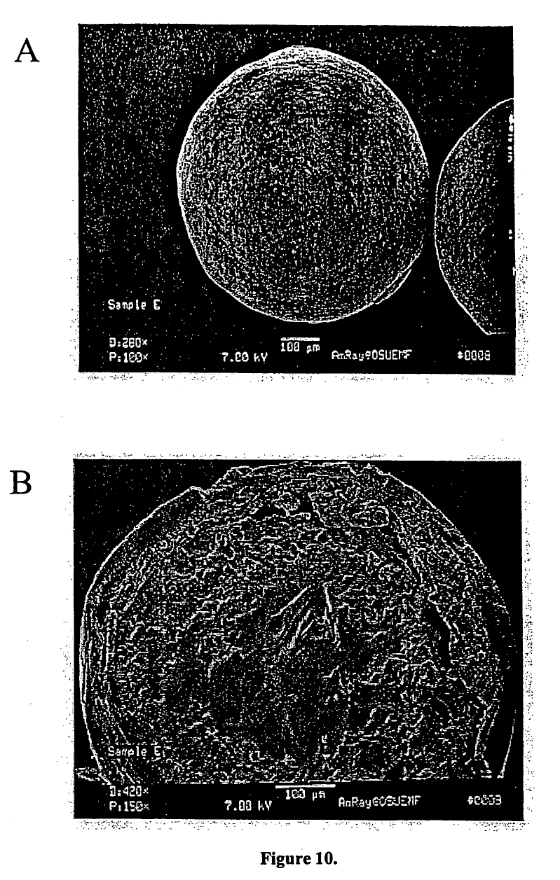

The surface of the enteric-coated Gelucire hot-melt coated beads containing RPE (Table 8) appeared to be highly uniform and compact (FIG. 10a), which is characteristic of aqueous-based methyl methacrylic acid copolymer film coating, but somewhat surprising given the ribbon-like unevenness of the direct blending hot-melt coating which is directly under the enteric coat. The cross-section view (×150) clearly indicated good coalescence of the enteric coating material (FIG. 10b). The hot-melt coating layer again is not visible in FIG. 9b. There appeared however to be an apparent separation at the nonpareil and the enteric-coating layer interface, which was manifested from the SEM sample preparation. The disjoining feature between the two layers may be the result of re-melting of the hot-melt coating layer during the drying step after enteric coating. Initially when the enteric-coating layer was applied, the hot-melt layer remained in intimate contact with both the nonpareil surface and the enteric film layer since Gelucire was already in semi-solid state at the temperature involved. As soon as the enteric coating had been fully applied, and then heating began, the hot-melt applied coat could melt since the process temperature was allowed to vary from 40-50° C., which is in the melting point range (46-51° C.) for Gelucire® 50/13. This re-melting process may destroy the semi-solid structure (make it molten again) of the Gelucire hot-melt layer during complete drying of the enteric coating, which is not problematic because the melting Gelucire is trapped inside the now present enteric coating “overcoat”. Re-congealment of molten Gelucire during final cooling of the enteric coating process may have left gaps between the enteric-coating layer and the nonpareil surface that were previously filled with Gelucire® 50/13 coating material.

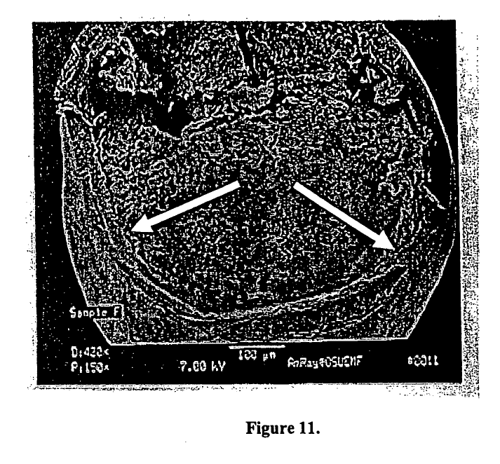

Cross-section view of enteric-coated Gelucire hot-melt coated nonpareil suggested the existence of re-melting and re-congealment for Gelucire during enteric coat drying, by the disconnection between the enteric coating layer and the nonpareil surface (FIG. 11).

Although some surface irregularity was seen in individual beads, it is an important observation that the batch of beads had a uniform drug content that was not expected.

### Example 8

### Influence of Drug Addition on Hot-Melt Agent Melting Point Using Thermal Analysis

Differential scanning calorimetry (DSC) thermograms for Gelucire® 50/13 and mixture of Gelucire® 50/13 to Ragweed Pollen Extract (RPE) in a 5:1 ratio were generated using Perkin-Elrner DSC-4/Thermal Analysis Data Station (TADS) System in sealed aluminum pans. The heating rate was 20° C./min. The thermogram for Gelucire was obtained from a Gelucire® 50/13 sample as packaged. The thermogram for the mixture of Gelucire and RPE was acquired from a congealed Gelucire-RPE sample. The congealed sample was prepared by melting Gelucire, dispersing RPE into the molten Gelucire, and finally congealing of the molten material. The DSC technique was utilized to study the effect of RPE addition on Gelucire melting point and also the rate for molten Gelucire congealing.

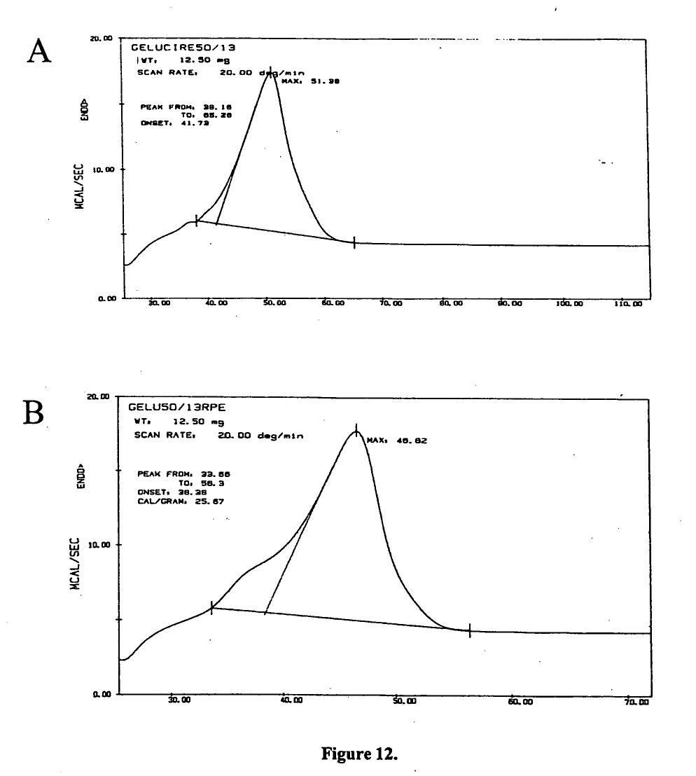

Differential scanning calirometry (DSC) revealed that the addition of RPE depressed Gelucire® 50/13 average melting point from 51.38° C. to 46.62° C. (FIG. 12). This is indicative of the formation of a eutectic mixture. The reduced average melting point for Gelucire® 50/13 also means lowered congealing point when it starts to solidify. This teaches that melt coating materials can remain in molten state for extended period of time at lower temperature when containing RPE. For thermally sensitive drugs, this unexpected lower congealing temperature would mean improved stability since the processing temperature can be reduced accordingly.

Lower processing temperature with this new direct blending melt coating method provides energy savings, time savings and other processing and stability advantages for all pharmaceutical agents. While typical spray melt coating methods require that the melt coating material be maintained at a temperature of 40-60° C. above its melting point (Franz, supra) in order to prevent molten coating material from congealing prior to being delivered to the substrate surface, the coating for the new direct blending melt coating method is normally kept at about only 20-30 degrees above the melting point prior to application to the substrate surface, more preferably at 10-20 degrees above the melting point, and even more preferably at only about 5-15 degrees above the congealing point of the molten coating material optionally containing active agent and formulation ingredients. Thus, for example, a coating material with a melting point of about 50 degrees centigrade may require a molten temperature of as much as 90-110 degrees centigrade for traditional spray melt coating with a fluid bed method but the same coating may be successfully applied in some cases at only about 60 degrees centigrade or less using the new method disclosed herein. Drug degradation during product production must be minimized and even a few degrees can make a difference in drug stability. And, with spray melt coating in a fluid bed, those few degrees may make a difference in ability to conduct the process.

### Example 9

### Description of Hot-Melt Coating of Capsule by Direct Blending Method

The hot-melt coating procedure illustrated in FIG. 2 for coating nonpareils of 10-40 mesh was also applied for hot-melt coating of an empty capsule (average 49 mg/capsule; capsule size:3CS; Color: natural transparent 0000; Capsugel®, Division of Warner-Lambert Company, Greenwood, S.C.). However, the capsule preheating step (Stage 3 in FIG. 2) was not utilized for the capsule coating as it was for the nonpareil coating. Multi-layer hot-melt coating of capsule can also be carried out with the subsequent layers of hot-melt coating being applied to the already-coated capsules. Upon completion of one layer of coating, the coated capsules were not screened through any size of sieve before proceeding to the next layer of hot-melt coating.

### Example 10

### Floating Study of Hot-Melt Coated Capsule

In one floating study, the hot-melt coated capsules were placed first in simulated gastric fluid (SGF) for 2 hours and then transferred over to simulated intestinal fluid (SIF) in a covered 200 ml beaker at room temperature. A second floating study was performed only in SIF but at 37.5° C., which was maintained in a computer-controlled water bath. All floating studies were conducted under static condition, i.e., without medium movement in the beaker.

The floating study was designed to investigate preliminarily the floatability of the hot-melt coated capsule as a potential gastric retention device for controlling drug release.

Prior to including stearic acid into melt coat preparation, the Gelucire 50/13 hot-melt coated capsules (after 8 coating layers of about 2000% total weight gain) were able to float for about 2 hours in simulated gastric fluid (SGF) before Gelucire 50/13 coating layer was dispersed into the medium, exposing the capsule surface. Exposed capsule shells would then dissolve, the capsule filled with liquid, and sank. After coating the existing capsules with another layer (about 250% in weight gain) of Gelucire 50/13/stearic acid (5:1) hot-melt mixture, the capsules were surprisingly found to float for about 5 hours before the melt coat was dispersed into the medium. The increased floating time was mainly due to the water repellant property of stearic acid incorporated into the hot-melt mixture matrix. Capsules coated with either Gelucire 50/13 or mixture of Gelucire 50/13/stearic acid (5:1) underwent minimal agglomeration.

As a result of this observation, a mixture of Gelucire 50/13/stearic acid (5:1) was selected for capsule hot-melt coating and the subsequent floatability study. Table 11 describes the capsule hot-melt coating formulation for the first coating layer. For multi-layer coating, the percentages for the hot melt materials would be the same for each subsequent layer. The number of capsules (size 3CS) was about 90-100 for the coating formulation described in Table 11.

TABLE 11
Capsule hot-melt coating formulation for first layer coating.

| Materials | Weight (g) | % (hot melt materials) |
|---|---|---|
| Capsule (3CS) | 4.6 | — |
| Gelucire 50/13 | 5* | 83.33 |
| Stearic acid | 1* | 16.67 |

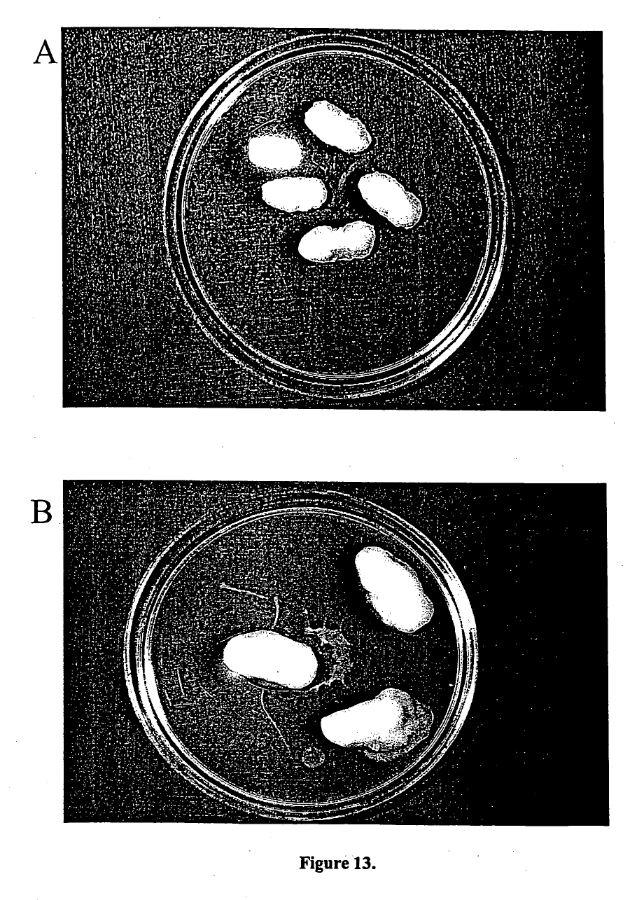

Capsules coated with only Gelucire 50/13/stearic acid (5:1) layer (about 1000% in weight gain through multi-layering) were able to float in simulated gastric fluid for 2 hours before being transferred over to simulated intestinal fluid, where the capsules then floated for more than 70 hours. Clearly, the capsules would float for an extended time with much less coating weight gain. Over the duration of the entire 70-hour floating process, Gelucire50/13/stearic acid layer was observed to swell in all directions to a size about 1.5-2 times the original capsule volume. Only one half of the coated capsule was immersed into the medium while the second half was above the medium surface. Even with the swelling effect, Gelucire50/13/stearic acid layer was not observed dispersing into the medium. FIG. 13 compares coated capsules before and after floatability study. It is expected that the floating/swelling as shown results in a capsule with extended gastric retention time.

As can be seen from b in FIG. 13, Gelucire 50/13/stearic acid layer still retains most of its original shape after 16 hours of floating, although the outmost layer surface seems to be much softer than the inner part. Since the coated capsules are able to float in both simulated gastric fluid and simulated intestinal fluid, it can be discerned that the hot-melt coating is not influenced significantly by pH of the media used.

Gelucire 50/13/stearic acid hot-melt coated capsules of the same batch were also investigated for floating in simulated intestinal fluid at 37.5° C. under static conditions. It was observed that the capsules were also able to float for more than 12 hours (when observation was stopped) with minor hot-melt dispersion of the coating surface into SIF at the bottom of the container. The coated capsules were not found to be as solid as those studied at room temperature after 16 hours of floating. This experiment shows that the melt coating method can be applied to capsules as a pharmaceutical substrate with optional multiple coatings in unexpected amounts producing as much as 2000 percent weight gain with minimal substrate agglomeration. At least one of the molten coating materials or the pharmaceutical substrate may contain an active pharmaceutical agent. Materials such as fatty acids or other substances known to influence gastric emptying time may be included in the coating material on an empty capsule (contains air) that will float in the stomach following oral administration and provide sustained delivery of the active agent from the coating material. Natural swelling of the coating materials can be promoted by adding expandable hydrogels such as hydroxy propyl methyl cellulose, methyl cellulose, and the like. The capsule may also be partially filled or even full with placebo or active agent formulation and still float as a result of entrapped air and the very low density (high buoyancy) and non-wetting of fatty acid materials in the coating. Filling or partial filling, along with active agent formulation if desired, may take place with helium or other lighter-than-air pharmaceutically acceptable gas to entrap the gas inside the capsule and the melt coating method applied in a helium or lighter than air environment. The gas will help the capsule float and any pharmaceutically active agent present may leak into the stomach or be delivered in the intestine after the capsule leaves the stomach. Such formulations are valuable as gastric drug delivery devices, for sustained controlled release of drugs, and for appetite suppression or as weight loss aids.

### Example 10A

### Melt Coating of Drug on Capsules

Gelatin capsules size 4 (average weight 38 mg) were melt coated with the drug Verapamil HCl using the general method of Example 9. Gelucire 50/13 (melting point 50 degrees centigrade) was melted at 65 degrees centigrade and then cooled to 60 degrees centigrade. Verapamil HCl was dispersed into the molten Gelucire in a 1 part drug to 1 part Gelucire ratio with stirring and then empty size 4 gelatin capsules were added into the molten mixture and stirred, and after uniform blending the coated capsules were allowed to cool to room temperature with continuous stirring while the coating/drug mixture congealed on the capsules. This process was repeated until the total weight gain of coating containing drug for the empty capsules was 200% (76 mg of coating/drug mixture on 32 mg capsules). Dissolution studies in a USP paddle stirring apparatus at 50 RPM in simulated gastric fluid without enzymes at 37.5 degrees centigrade resulted in 66% drug dissolved in 0.5 hours and 97% drug dissolved in one hour. The capsules floated on top of the gastric fluid during the entire drug release period.

Verapamil HCl is a phenylalkylamine derivative that antagonizes calcium influx through slow channels of vascular smooth muscle and cardiac cell membranes. It is useful in treatment of angina, cardiac arrhythmias and hypertension. This water-soluble drug provides another example that pharmaceutical agents can be formulated using the newly disclosed hot-melt coating by direct blending. Drugs with lower solubility and different physical-chemical characteristics along with different melt-coating excipients can be used with the new method and will release drug from the formulations at different rates. One skilled in the art will readily understand that varying melt-coating excipients and drug solubility programs desired rates of pharmaceutical agent release.

### Example 10B

### Melt Coating of Partially Filled and Filled Capsules

The new hot-melt coating by direct blending method was used to prepare 4 different oral pharmaceutical formulations with verapamil HCl as the pharmaceutical agent and partially filled or filled gelatin capsule as the pharmaceutical substrate. Gelatin capsules size 4 were partially filled with either 40 mg of verapamil HCl or filled by hand packing with a homogeneous mixture of 40 mg of verapamil HCl and 100 mg of lactose. These pharmaceutical substrates were then coated according to the process described above and the ingredients shown below.

| | | Molten coating mixture | Molten |
|---|---|---|---|
| Formu- | Contents of | applied | overcoating mixture |
| lation | capsule | Per capsule | Applied per capsule |
| A | 40 mg drug | Gelucire 50/13 (21 mg) | None |
| | | and drug (40 mg) | |
| B | 40 mg drug | Gelucire 50/13 (21 mg) | 100 mg mixture of |
| | | and drug (40 mg) | Gelucire 50/13 |
| | | | with stearic acid (5:1) |
| C | 40 mg drug | Gelucire 50/13 (21 mg) | None |
| | 100 mg | and drug (40 mg) | |
| | lactose | | |
| D | 40 mg drug | Gelucire 50/13 (21 mg) | 200 mg mixture of |
| | 100 mg | and drug (40 mg) | Gelucire 50/13 |
| | | | with stearic |
| | | | acid (5:1) lactose |

Temperature of molten coating with a mixture of gelucire and stearic acid was maintained at 10 degrees centigrade above the congealing point during mixing with the pharmaceutical substrate.

Formulation A and C are immediate release oral dosage forms as shown using USP dissolution apparatus II in simulated gastric fluid at 37 degrees centigrade and paddle stirring (50 RPM). Drug dissolved in gastric fluid was 77% (A) and 79% (C) at 0.25 hours, and complete at 0.75 hours for both oral dosage forms. Dissolution results for B and D are shown below.

Percent Drug Dissolved

| | Time (hours) | | | | | | | | | | | | | | | |
|---|---|---|---|---|---|---|---|---|---|---|---|---|---|---|---|---|
| | .08 | .17 | .25 | .5 | .75 | 1 | 1.5 | 2 | 3 | 4 | 5 | 6 | 7 | 8 | 9 | 10 |
| Formulation B | <3 | <3 | <3 | <3 | <3 | 3.6 | 6.6 | 19.1 | 52.6 | 70 | 90 | | | | | |
| Formulation D | <3 | <3 | <3 | <3 | <3 | <3 | <3 | <3 | <3 | 23.0 | 62 | 72 | 86 | 87 | 89 | 91 |

Floating time for the capsules was 16-20 minutes for Formulation B and 5.5-6 hours for Formulation D.

These data are consistent with Example 10 and show prolonged floating of the dosage form in gastric fluid and a highly desirable programmed drug release pattern of a lag time from about 1.5-2 hours for Formulation B and about 3.5 hours for Formulation D followed by sustained release of drug until about 5-6 hours for Formulation B and until 10 hours for Formulation D. This release pattern is appropriate for, among others, antihypertensive drugs because blood pressure follows a circadian rhythm pattern and spikes in the early morning in correlation with an increase in heart attacks in the morning hours. Thus, taking antihypertensive drugs at 9-11 PM in a dosage form that has a lag time of 2 or more hours followed by controlled sustained release provides appropriate drug concentration in the body when it is most needed, during the early hours of the day. One skilled in the art will readily recognize that the new hot-melt coating by direct blending method demonstrated herein is readily adaptable to any pharmaceutical agent with acceptable stability in the method and known excipients and ingredients to control drug release from pharmaceutical dosage forms in a variety of patterns within the range presented herein and drug release can be sustained until 12 or 24 or even beyond 70 hours if desired.

### Example 10C

### Coating Drug Loaded Capsules

Size 4 gelatin capsules were filled with a mixture of 80 mg of verapamil hydrochloride and 60 mg of lactose. These capsules were then hot-melt coated by direct blending with a mixture of 5 parts of gelucire (50/13) to one part of stearic acid. The composition produced was a waxy coating material with a melting point between 50 and 60 degrees centigrade on coated discrete substrate capsules. Three compositions were produced with coating weight gains of 150 mg, 200 mg, and 250 mg. The following table gives dissolution results in simulated gastric fluid at 37 degrees centigrade with paddle stirring at 50 RPM

Percent Drug Dissolved

| | Time (hours) | | | | | | | | | | |
|---|---|---|---|---|---|---|---|---|---|---|---|
| | 1 | 1.8 | 2 | 2.3 | 2.5 | 2.75 | 3 | 3.8 | 4 | 4.2 | 4.5 |
| Coating | 2 | 50 | 84 | 97 | | | | | | | |
| 150 mg | | | | | | | | | | | |
| Coating | 1 | 13 | 35 | 64 | 88 | 98 | | | | | |
| 200 mg | | | | | | | | | | | |
| Coating | | | 4 | | | | 5 | 32 | 51 | 72 | 97 |
| 250 mg | | | | | | | | | | | |

Floating time for capsules with a 250 mg coating of this particular coating material was about 3.5-4 hours, and then the capsules sank for about 1-2 hours, and then the empty coating/shell began to float again, and then sank again after another 1-2 hours.

This example shows a discretely coated pharmaceutical substrate with a waxy coating material that has a waxy feel and is easy to swallow. Other pharmaceutical substrates such as tablets, beads or granules can also be discretely coated using the new method. Lag time before drug is released is controlled by the amount and materials of coating applied. This example, in combination with example 10 and 10A shows that a “pulse-type” drug release pattern can be produced using the new method. Immediate release drug in a melt coating are overcoated on the capsules of Example 10B (above) to provide an initial pulse of drug release, and then the core contents of the capsule provide a delayed pulse release of drug following a lag time. Note that once drug starts to be released from the coated substrate of Example 10B, the release is rapid and is nearly complete within about 2 hours or less. It is expected that substrates other than capsules would achieve a similar lag time. Such compositions are useful in drug delivery, e.g., like amphetamines and methylphenidate used in treatment of hyperactive children or administration of melatonin in phase shifting or replacement therapy.

### Example 11

### Immunogenicity Study in Näive Mice of Enteric-Coated Ragweed Pollen Extract (RPE) Alpha Fraction Prepared by Direct Blend Hot-Melt Coating Method of the Invention

Immunogenicity study results showed that Ragweed Pollen Extract (RPE) encapsulated into microbeads by both a spray coating method (spray coat formulation) and a new hot-melt coating by direct blending method (direct blend formulation) was able to induce an equivalent average anti-RPE antibody response in responder mice after being administered orally. This experiment clearly shows one value of using the method of melt coating a pharmaceutical substrate comprising submerging the substrate in a molten coating material wherein the molten coating material contains a heat sensitive antigen. The antibody titers of responders in both the treatment groups (dosed with RPE microbeads prepared either with conventional spray coat formulation or the direct blend formulation) were found to be statistically different from those of the placebo group. However, there was no significant difference found between the two treatment groups. An ELISA Inhibition assay also demonstrated that the RPE, encapsulated by either formulation, retains its native conformational form.

In order to assess the ability to generate an immune response by allergen encapsulated using the new formulation, it was desirable to compare the response elicited by allergen encapsulated by the Wurster technique described in a patent (U.S. Pat. No. 5,591,433) and by the inventive formulation. An immunogenicity study (placebo-controlled) was designed and conducted in mice using ragweed pollen extract (RPE) as the model allergen in both the original and new process. Mice are an animal model well suited to demonstrating immunogenicity of proteins. It was shown that mice immunized with RPE produce antibodies against Amb a 1, the major allergenic protein in RPE recognized by the sera of humans with RPE allergy.

A placebo was prepared by employing the direct blend formulation of the invention with the absence of RPE. The final enteric-coated particles will be referred to as microbeads.

Materials and Methods

Animals

Female, 8-12 week old Bald/c mice were obtained from and kept at the animal facility of B&K Universal (Fremont, Calif.). The mice were housed in polycarbonate cages with stainless steel wire tops. Each cage was covered by a microisolator hood to prevent cross contamination. Mice were acclimated for a minimum of 3 days at B&K Universal prior to the start of the study.

Allergen Encapsulation

Standardized, lyophilized short ragweed Alpha Fraction was prepared by Hollister-Stier Laboratories, LLC (Lot#: AF00200T; Spokane, Wash.). The ragweed Alpha fraction is a diafiltered extract lacking the very low molecular weight components (<3,000 MW) of the ragweed extract. The reported potency of the RPE raw material is 36.6 Amb a 1 Units/mg.

For encapsulation by the spray coat formulation, RPE was solubilized in a solution containing lactose (NF, monohydrate) (Ruger Chemical Co., Inc., Irvington, N.J.) and polyvinylpyrrolidone (Plasdone® K29/32) (ISP Technologies, Inc., Wayne, N.J.) as a binding agent. The solution was spray-coated onto nonpareils of 30-35 mesh (CHR Hansen, Wahwah, N.J.) in a fluidized bed with Wurster bottom insert (Fluid Air, Aurora, Ill.). Following application of the RPE layer, the particles were enteric-coated with an aqueous methyl methacrylic copolymer dispersion (Eudragit L30D 55S, Rohm America Inc, Somerset, Mass.). All processes were conducted at low temperatures in order to minimize possible protein degradation. For encapsulation by the direct blend formulation, RPE was incorporated onto nonpareils via hot-melt coating by direct blending method of the invention. Following application of the RPE layer, the particles were enteric-coated as with the spray coat formulation but based on processing conditions described in Table 3.

The potencies of RPE microbeads were analyzed to be 205 Amb a 1 Units/gram of finished beads and 139.4 Amb a 1 Units/gram of finished beads, respectively by the conventional spray coat formulation and the direct blend formulation of the invention. Enteric-coated RPE particles are acid-resistant at pH 1.2 for at least 2 hours and readily become solubilized at pH 5.5 or above, releasing RPE proteins, as assessed by the BCA (bicinchoninic acid) protein assay (Pierce, Rockford, Ill.). In other words, encapsulated RPE is protected from gastric contact in the stomach and is released when reaching the small intestine where the pH is suitable for dissolution of the enteric layer. The placebo microbeads were also prepared according to the direct blend formulation with the absence of RPE proteins.

Mental Design for Mice Immunization

Mice were randomly divided into three immunization groups with 15 mice in each group. One group received RPE microbeads with a potency of 205 Amb a 1 Units/gram of finished beads; the second group received RPE microbeads with a potency of 139.4 Amb a 1 Units/gram finished beads; the third group received the placebo beads. The study design is described in Table 12 along with the study schedule in Table 13.

TABLE 12
Experimental design for the mice immunogenicity study

| | | | Total | |
|---|---|---|---|---|
| | | | Amb | |
| Group | | | a 1 | Number |
| Number | Group Designation | Dosage | Units | of Mice |
| I | RPE microbeads by | 16.5 mg RPE | 9.2 | 15 |
| | direct blend | microbeads/mouse | | |
| | formulation (139.4 | for 4 consecutive | | |
| | Amb a 1 Units/gram | days | | |
| | beads) | | | |
| II | Placebo microbeads by | 16.5 mg placebo | 0 | 15 |
| | direct blend | microbeads/mouse | | |
| | formulation | for 4 consecutive | | |
| | | days | | |
| III | RPE microbeads by | 15 mg RPE | 9.2 | 15 |
| | spray coat formulation | microbeads/mouse | | |
| | (205 Amb a 1 | for 3 consecutive | | |
| | Units/gram beads) | days | | |

TABLE 13
Mice immunogenicity study schedule

| Study day | Events |
|---|---|
| 0 | Pre-treatment: blood draw from each mouse |
| 1 | Oral microbead dosing of all groups |
| 2 | Oral microbead dosing of all groups |
| 3 | Oral microbead dosing of all groups |
| 4 | Oral microbead dosing of groups I and II |
| 15 | Blood draw from all groups |
| 22 | Blood draw from all groups |
| 23 | Oral microbead dosing of all groups |
| 24 | Oral microbead dosing of all groups |
| 25 | Oral microbead dosing of all groups |
| 26 | Oral microbead dosing of groups I and II |
| 36 | Blood draw from all groups |
| 50 | Blood draw from all groups |
| 64 | Blood draw from all groups |
| 71 | Euthanize all mice on study |

Study Procedures

Mice are dosed under light anesthesia (isofluorine) with microbeads delivered to the back of the oral cavity using a feeding device developed by Allergenics, Inc. Each animal receives a drop of acidified water to facilitate swallowing of the microbeads. Blood samples are taken from the retro-orbital plexus in mice that are anesthetized with isofluorine using heparinized capillary tubes. Two hundred microliters of blood were drawn from each mouse and alternate plexus sites were used for consecutive sampling. Sera obtained from blood draw was diluted in sterile PBS (pH 7.4) in a 1:2 ratio. The diluted blood sample was centrifuged to pellet the red blood cells and the serum collected stored at 4° C. for assay.

Serum Antibody Assay

Serum anti-RPE antibody titers were determined by a direct ELISA assay. Microtiter plates (96-wells) (NUNC Immuno™ plate, Nalge NUNC International, Denmark) were coated with RPE dissolved in pH 9.6 carbonate buffer (0.1 mg/ml, 100 μl per well). After overnight incubation at 4° C., the plates are washed once with PBS/Tween and filled with 150 μl/well of 10% FBS in PBS/Tween to columns 1 to 10, followed by an extra addition of 120 μl/well to row A only. Sample sera (from mouse) of 30 μl were added to wells in row A in the plates followed by a serial titration of 150 μl from row A through row G. After incubation at 4° C. for one hour, the plates were washed three times with PBS/Tween and filled with 100 μl/well of 10% fetal bovine serum (FBS) in PBS/Tween containing goat anti-mouse IgG (H+L) HRP (1 mg/ml, Zymed, South San Francisco, Calif.) at a concentration of 1:2000. After incubation at 4° C. for one hour, the plates were washed three times with PBS/Tween. The serum samples were developed by adding 100 ml/well of Tetramethyl Benzidine (TMB) (Zymed, South San Francisco, Calif.) to the plates to yield a blue color. To stop the color reaction, 100 μl/well of 0.5M H₂SO₄ (Fisher, Pittsburgh, Pa.) were added to the plates to yield a yellow color.

In each of the plates analyzed, a standard curve was prepared using pooled serum samples from all mice in the two treatment groups (Group I and III, Table 12) at the scheduled blood-draw on day 50 (Table 13). Serum from this date was chosen to provide maximum antibody titers based on assay results from previous studies. The assay procedure used for the standard curve preparation was the same as that for the serum sample from individual mice. The Microtiter plates were analyzed using a microplate reader (Multiskan MCC, MTX lab system, Inc., Vienna, Va.) at wavelength of 450 nm.

Quantification of Serum Antibody Titers

An arbitrary antibody titer of 1,000 was assigned to the most concentrated pooled serum sample of the averaged standard curve. Antibody titration of this antisera 1:2 on the ELISA plate resulted in wells with standard titers of 500, 250, 125 and so on. An equation relating absorbance at 450 nm and antibody titer was determined by linear regression analysis. The serum anti-RPE antibody titers were estimated for each sample in the following fashion:

For example, the standard equation between the antibody titer and the absorbance for one plate analyzed is:
Antibody titers=111.45×Absorbance−34.965

Assume that the absorbance readings from a serum sample titration (1:2) are 1.155, 0.595, and 0.341. The actual antibody titer values can therefore be estimated according to:
Actual antibody titers=[111.45×Absorbance−34.965]×dilution_factor
The actual antibody titers are then approximated to be 93.76 (93.76×1), 62.70 (31.35×2), and 12.16 (3.04×4), respectively. As a result, the antibody titer for this particular serum sample is estimated to be 78.23 [(93.76+62.70)/2]. The last antibody titer (12.16) was excluded from the calculation since it was drastically different from the first two titers.
Statistical Analysis

Statistical differences among the values associated with the experimental groups are determined using the two-sample, 2-tailed t-test (assuming equal variance). The values of P<0.05 are considered to be statistically significant and are denoted with asterisks in the FIG. 17.

Results and Discussion

Validation of Pooled Serum Sample for Standard Curve Preparation

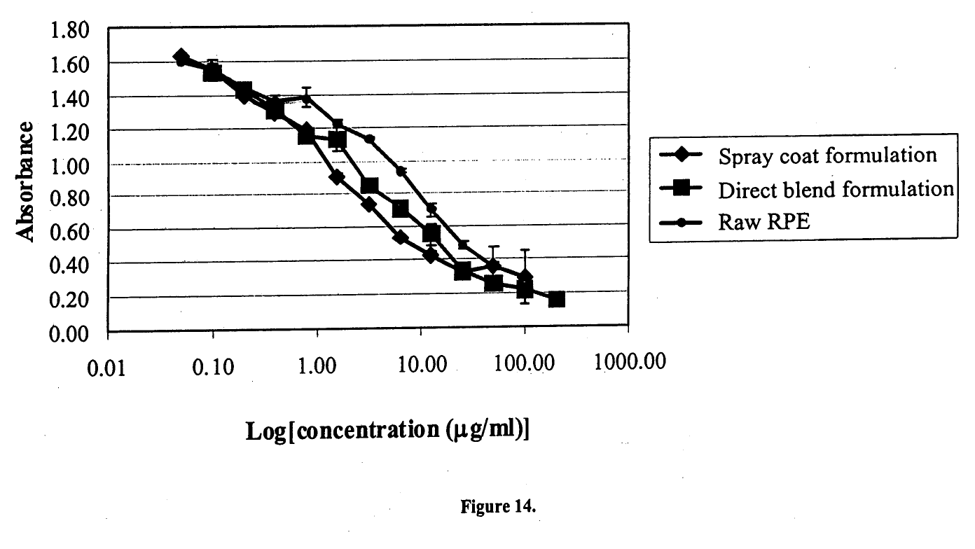

The ELISA Inhibition assay (different from direct ELISA assay employed for serum antibody analysis) results demonstrated that the anti-RPE antibodies from the pooled serum reacted similarly to the RPE-inhibitor released from both enteric-coated preparations (FIG. 14). The equivalent ability of RPE released from direct blended microbeads and RPE released from spray coated microbeads is reflected by the similar absorbance vs. RPE inhibition curve as depicted in FIG. 14. As a result, the utilization of pooled serum sample for the standard curve preparation becomes valid.

The absorbance reading from ELISA Inhibition assay is an indirect measurement of the binding between the anti-RPE antibodies and the RPE-inhibitor. As the RPE inhibitor solutions become more concentrated, a greater amount of anti-RPE antibody binds to the inhibitor rather than the ELISA plate. The inhibitor bound antibody is washed from the plate during the ELISA assay. FIG. 14 showed that the absorbance appears to be inversely proportional to the log of the RPE inhibitor concentration. The inhibition curves are identical whether the inhibitor is from the spray coat formulation, the direct blend formulation, or from the raw RPE material.

Examination of FIG. 14 also showed that the slopes for all three of the profiles are strikingly similar to each other. This occurrence implies that the anti-RPE antibodies from the pooled serum sample are binding in a comparable fashion to the RPE-inhibitor complexes from each of the three categories. Given that the anti-RPE antibodies and the RPE inhibitors employed are identical in each of the three profiles, it can be concluded that RPE must exist in the same form across all three categories. Since raw RPE exists in its native conformational form, the encapsulated RPE also remains in its native conformational structure following preparation by both the spray coat formulation and the direct blend formulation of the invention that involves mixing into hot melt coating.

Induction of an Immune Response in Mice after Oral Administration of Encapsulated Ragweed Pollen Extract (RPE)

Over the course of the study, a number of mice perished from each of the three groups, which might have been caused by dosing since it is difficult to intubate mice. The serum samples were collected and analyzed from mice that survived at each of the six scheduled blood-draws.

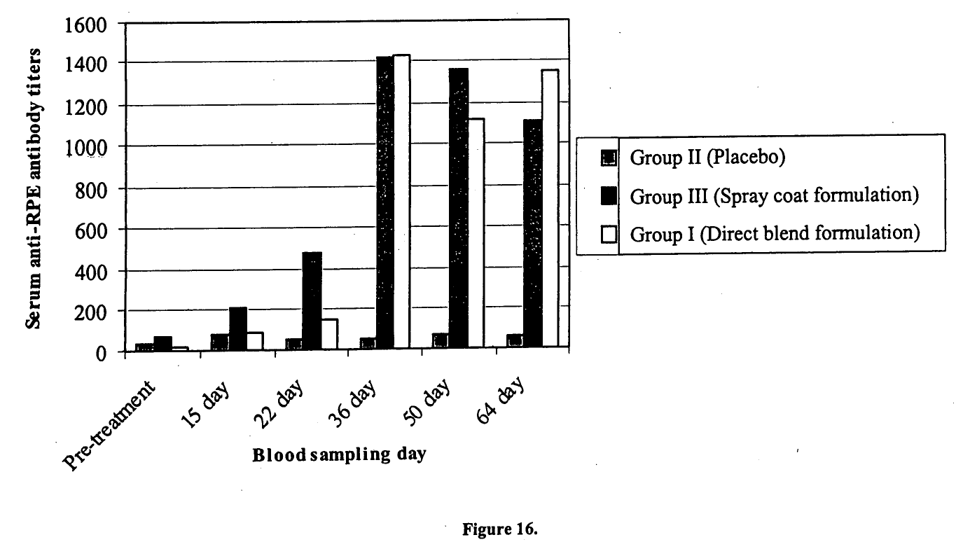

FIG. 16 depicts the average total serum anti-RPE antibody titers for the 3 treatment groups. As can be seen, the trend for the antibody responsiveness is initially the most dramatic for Group III that was dosed with RPE encapsulated microbeads by the spray coat formulation. The antibody response peaks at around the 36^{th} day into the study followed by moderate decline thereafter. The antibody response for Group I (dosed with RPE encapsulated microbeads by the direct blend formulation) is also apparent however with a slower rate of increase over time. This response peaks at around the 36^{th} day into the study then is followed by moderate reduction and stabilization in antibody response, and is very similar to the spray coat formulation at the peak (day 36) and thereafter. As expected, Group III (the placebo group) failed to demonstrate an anti-RPE antibody response, which are reflected by the stable base-line reading throughout the duration of the investigation.

TABLE 14
Statistical analysis comparing the treatment groups and the control
group for serum anti-RPE antibody titers after oral administration of
RPE encapsulated enteric-coated microbeads.

| Comparison | 2. P-value* | | | | | |
|---|---|---|---|---|---|---|
| between | Pre- | | | | | |
| groups | treatment | 15-day | 22-day | 36-day | 50-day | 64-day |
| Group III | 0.16 | 0.20 | 0.17 | 0.70 | 0.38 | 0.69 |
| vs. Group I | | | | | | |
| Group III | 0.27 | 0.48 | 0.21 | 0.11 | 0.07 | 0.10 |
| vs. Group II | | | | | | |
| Group I vs. | 0.14 | 0.13 | 0.47 | 0.19 | 0.25 | 0.25 |
| Group II | | | | | | |

*P-values <0.05 are considered to be significantly different.

TABLE 15
The average and the standard deviation of the serum anti-RPE antibody titers for the
treatment groups (Groups I and III) and for the control group

| Treatment | Statistics on | Pre- | | | | | |
|---|---|---|---|---|---|---|---|
| Group | Titers | treatment | 15-day | 22-day | 36-day | 50-day | 64-day |
| Group I | verage* | 21 | 48 | 81 | 602 | 472 | 477 |
| (direct blend | StDev** | 13 | 46 | 102 | 1058 | 822 | 834 |
| formulation) | | | | | | | |
| Group II | Adverage* | 30 | 73 | 55 | 52 | 65 | 59 |
| (Placebo) | StDev** | 15 | 28 | 26 | 31 | 32 | 25 |
| Group III | Average* | 48 | 116 | 245 | 794 | 829 | 626 |
| (spray coat | StDev** | 54 | 196 | 437 | 1140 | 945 | 784 |
| formulation) | | | | | | | |

*Average of the serum anti-RPE antibody titers from all mice in a group at scheduled blood-drawing day.
**StDev [=] standard deviation: of the serum anti-RPE antibody titers from all mice in a group.

Based on the averaged antibody titers observed with mice in the control group (row 2, Table 15), which are observed to fall below 100, mice in the treatment groups that show antibody titers less than 100 were considered non-responders and excluded from the analysis (those shaded in Table 16).

Analysis of RPE Responders for Antibody Titers

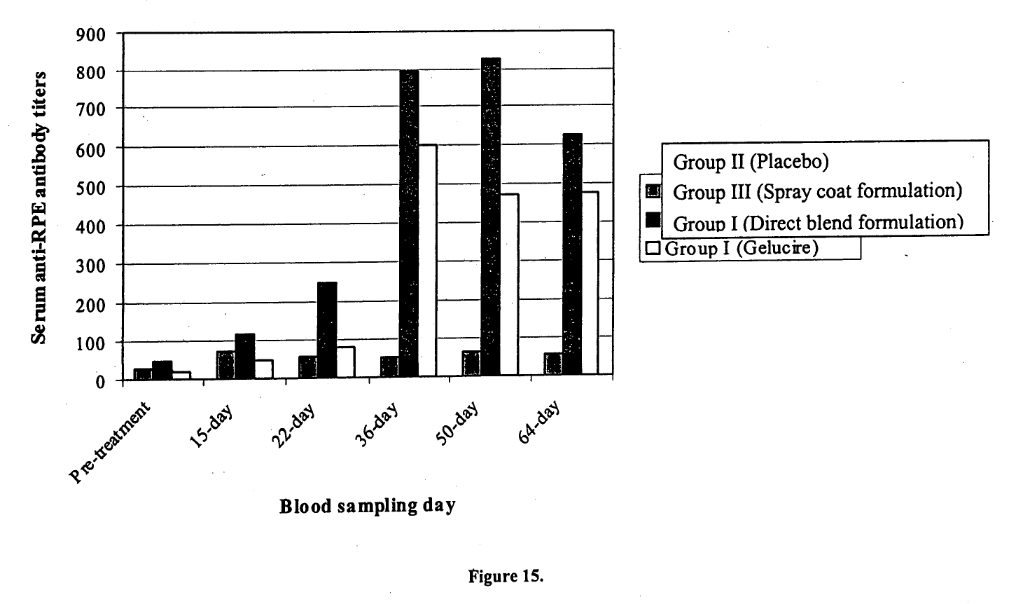

Analysis showed that the averaged serum anti-RPE antibody titers peaked at the same level around the 36^{th} day for both treatment groups I and III (FIG. 16). As also noticed in FIG. 16, the antibody response for group m appeared to be more uniform, indicated by steadier increase in the titer reading over the early duration of the blood sampling process. Given that mice in both groups were dosed with an equivalent amount of RPE, the difference in the rate of increase for the antibody response can possibly be explained by the fewer doses administered to group III (Table 13). Fewer doses were administered for this group since microbeads prepared by the spray coat formulation were higher in RPE potency (Table 12). There may exist a threshold dose required to trigger the onset of an antibody response, and the dose may be variable/mouse. The higher potency of the spray coated RPE microbeads may be above this “trigger” dose while the Gelucire microbeads are below. The comparison between FIG. 15 and FIG. 16 also shows that exclusion of mice in the two treatment groups that failed to respond to RPE produced a very similar antibody response profile for each of the treatment groups at the 36^{th}, 50^{th}, and 64^{th} day of blood-drawing.

Statistical analysis based on data used for FIG. 16 demonstrates significant differences between the placebo group (Group II) and each of the treatment groups I and III (Table 16). Due to the slower onset for the antibody response in Group I, the treatment difference became significant only after 36 days while for Group III, the treatment became significant at 22 days. As expected, there was no treatment difference found between Group I (dosed with microbeads prepared by the direct blend formulation) and Group III (dosed with microbeads prepared by the spray coat formulation) since the total amount of RPE administered was equal for all the mice in both groups, and non-responders were omitted.

TABLE 16
Summary of the total serum anti-RPE antibody titers for individual mice in
each of the treatment groups. The empty cells represent the absence of
blood samples due to the inability in blood collection and/or death of
mice. The bolded-text subjects are excluded for data analysis for
unresponsiveness to RPE.

| | | | | | | |
|---|---|---|---|---|---|---|
| SUBJECT # | pre-bleed | 15-day | 22-day | 36-day | 50-day | 64-day |

Group I (direct blend formulation)

| | | | | | | |
|---|---|---|---|---|---|---|
| 462 | 31.9 | 21.1 | 27.4 | 32.4 | 41.6 | 27.8 |
| 463 | 49.5 | 38.9 | 80.1 | — | — | — |
| 464 | — | 42.9 | 22.8 | 33.9 | 558.6 | 40.9 |
| 465 | 18.4 | 19.7 | 25.7 | 2026.1 | 608.3 | 1013 |
| 466 | 12.9 | 20.1 | 14.4 | — | — | — |
| 467 | 25.2 | 23.5 | 28.3 | 45.3 | 34.4 | 39.7 |
| 468 | 9.6 | 32.6 | 62.1 | 628.1 | 615.5 | 543.4 |
| 469 | 17.9 | 175.6 | 278.4 | 3031.3 | 2690.7 | 2507.7 |
| 470 | 5.2 | 9.9 | 28.8 | 20.8 | 18.2 | 24.9 |
| 471 | 14.9 | 29.3 | 95.7 | 70.9 | 49.9 | 51.2 |
| 472 | — | 23.9 | 27.9 | 56.6 | 29.3 | 47.7 |
| 473 | — | 38.8 | 96.6 | — | — | — |
| 474 | — | 36.6 | 36.7 | 76.7 | 72.1 | — |
| 475 | — | 128.3 | 360.6 | — | — | — |
| 482 | — | 83.8 | 24.4 | — | — | — |

Group III (spray coat formulation)

| | | | | | | |
|---|---|---|---|---|---|---|
| 448 | 8.6 | 103.6 | 72.5 | 3478.5 | 2353.3 | 1462.6 |
| 449 | 51.4 | 122 | 1348.7 | 1329.8 | 904.6 | 977.7 |
| 450 | 73.3 | — | — | — | — | — |
| 451 | 102.4 | 129 | 123.9 | 417.7 | 1549.6 | 850.9 |
| 452 | 178.6 | 722.6 | 955.6 | 2198.3 | 2313.5 | 2444.1 |
| 453 | 42.9 | 41.2 | 15.4 | — | — | — |
| 454 | — | 32.1 | 29.6 | 46.9 | 21.7 | 55.1 |
| 455 | 3.5 | 4.5 | 5.5 | 5.3 | 15.6 | 14.5 |
| 456 | 7.4 | 88.7 | 208.1 | 1009.1 | 876.6 | 790.8 |
| 457 | 18.5 | 26.1 | 16.3 | 42.6 | 69.7 | 78.3 |
| 458 | 35 | — | — | — | — | — |
| 459 | — | 6.2 | 10.5 | 7.7 | | 9.6 |
| 460 | 4.8 | 26.6 | 18.2 | 75.3 | 36.2 | 66.9 |
| 477 | — | 91.9 | 134.3 | 117.9 | 152.1 | 134.5 |

TABLE 17
Statistical analysis comparing the treatment groups and the control
group for serum anti-RPE antibody titers after oral administration
of RPE encapsulated enteric-coated microbeads. The data
employed for the analysis were only from mice in groups I and
III that showed antibody responses to RPE (non-bolded rows in
Table 16)

| Comparison | 1. P-value* | | | | | |
|---|---|---|---|---|---|---|
| between | Pre- | | | | | |
| groups | treatment | 15-day | 22-day | 36-day | 50-day | 64-day |
| Group III | 0.25 | 0.30 | 0.23 | 1.00 | 0.70 | 0.70 |
| vs. | | | | | | |
| Group I | | | | | | |
| Group III | 0.07 | 0.11 | 0.03** | 0.01** | 0.00** | 0.01** |
| vs. | | | | | | |
| Group II | | | | | | |
| Group I | 0.12 | 0.82 | 0.09 | 0.02** | 0.03** | 0.01** |
| vs. | | | | | | |
| Group II | | | | | | |

**P-values <0.05 are considered to be statistically different.

Thus, administration of Ragweed Pollen Extract (RPE) encapsulated into microbeads by the hot-melt coating by direct blending method was able to induce significant anti-RPE antibody titers in Balb/c mice. RPE microbeads prepared by both the direct blend hot-melt coating method and the spray coating method were shown to elicit equivalent anti-RPE antibody responses in the mice that responded. It was also demonstrated that RPE retains its native conformation following microencapsulation. This indicates that under the processing conditions imposed for encapsulation, RPE proteins were not degraded and potency was retained. Based on these results, it can be concluded that encapsulated RPE microbeads prepared by the hot-melt coating method is equivalent to those microbeads prepared by the spray coating method in their ability to induce an antibody response following oral administration. In addition, the hot-melt coating method may also be applied to other antigens as a feasible alternative to the spray coating method currently employed.

All of the references cited above are incorporated herein by reference.

The invention having now been described by way of written description and example, those of skill in the art will recognize that the invention can be practiced in a variety of embodiments and that the foregoing description and examples are for purposes of illustration and not limitation of the following claims.

1. A method of coating a pharmaceutical substrate, comprising:
providing a pharmaceutical substrate;
providing a molten coating material comprising less than 10% solvent; and
applying the molten coating material to the pharmaceutical substrate to form a coated substrate without fluidizing the pharmaceutical substrate with a gas stream while applying the molten coating material.

2. The method of claim 1 wherein the coated substrate comprises an antigen.

3. The method of claim 2 wherein the antigen is a heat sensitive antigen that is not degraded during the method.

4. The method of claim 2 wherein the molten coating material has a temperature of less than 40° C. above the melting point of the coating material when it is applied to the pharmaceutical substrate.

5. The method of claim 2 wherein the weight of the coating material in the coated substrate is greater than 6% of the weight of the pharmaceutical substrate in the coated substrate.

6. The method of claim 2 wherein the molten viscosity of the coating material is greater than 300 centipoise.

7. The method of claim 2 wherein the pharmaceutical substrate comprises beads smaller than 40 mesh.

8. The method of claim 2 wherein the antigen is uniformly dispersed in the coating material.

9. The method of claim 2 further comprising:
congealing the coating material on the pharmaceutical substrate; and
separating agglomerates before applying another coating to the coated substrate.

10. The method of claim 2 further comprising mixing the molten coating material during and/or after applying the molten coating material to the pharmaceutical substrate while the molten coating material is cooling and congealing on the pharmaceutical substrate.

11. (canceled)

12. The method of claim 2 wherein the pharmaceutical substrate is preheated before applying the molten coating material to the pharmaceutical substrate.

13. The method of claim 1 wherein the coated substrate comprises a pharmaceutical agent.

14. The method of claim 13 further comprising spray film coating the coated substrate.

15. The method of claim 13 wherein the pharmaceutical agent is a heat sensitive pharmaceutical agent that is not degraded during the method.

16. The method of claim 13 wherein the molten coating material has a temperature of less than 40° C. above the melting point of the coating material when it is applied to the pharmaceutical substrate.

17. The method of claim 13 wherein the weight of the coating material in the coated substrate is greater than 6% of the weight of the pharmaceutical substrate in the coated substrate.

18. The method of claim 13 wherein the molten viscosity of the coating material is greater than 300 centipoise.

19. The method of claim 13 wherein the pharmaceutical substrate comprises beads smaller than 40 mesh.

20. The method of claim 13 wherein the pharmaceutical agent is uniformly dispersed in the coating material.

21. The method of claim 13 further comprising:
congealing the coating material on the pharmaceutical substrate; and
separating agglomerates before applying another coating to the coated substrate.

22. The method of claim 13 further comprising mixing the molten coating material during and/or after applying the molten coating material to the pharmaceutical substrate while the molten coating material is cooling and congealing on the pharmaceutical substrate.

23. (canceled)

24. The method of claim 13 wherein the pharmaceutical substrate is preheated before applying the molten coating material to the pharmaceutical substrate.

25. A method of coating a pharmaceutical substrate, comprising:
providing a pharmaceutical substrate;
providing a molten coating material comprising less than 10% solvent;
applying the molten coating material to the pharmaceutical substrate to form a coated substrate,
wherein the weight of the coating material in the coated substrate is greater than 6% of the weight of the pharmaceutical substrate in the coated substrates.

26. The method of claim 25 wherein the coated substrate comprises a pharmaceutical agent.

27-42. (canceled)

43. The method of claim 1 further comprising spray film coating the coated substrate.

44. The method of claim 25 wherein the coated substrate comprises an antigen.
